(19) United States
(12) Patent Application Publication
(10) Pub. No.: US 2009/0264660 A1
(43) Pub. Date: Oct. 22, 2009

(54) PROCESS FOR ALKYL ARYL SULFIDE DERIVATIVES AND NEW SULFIDE COMPOUNDS

(75) Inventors: Heonjoong Kang, Kyeongki-do (KR); Jungyeob Ham, Seoul (KR); Jaeyoung Ko, Seoul (KR)

Correspondence Address: LOWE HAUPTMAN HAM & BERNER, LLP, 1700 DIAGONAL ROAD, SUITE 300, ALEXANDRIA, VA, 22314, US

(73) Assignee: Heonjoong KANG, Seoungnam-city, (KR)

(21) Appl. No.: 11/577,747

(22) PCT Filed: Oct. 21, 2005
(86) PCT No.: PCT/KR05/03528

§ 371 Date: Jun. 29, 2009

(30) Foreign Application Priority Data
Oct. 22, 2004 (KR) 10-2004-0085011
Oct. 21, 2005 (KR) 10-2005-0099926

(51) Int. Cl. C07D 405/12; C07C 321/28; C07D 317/18; C07C 323/36; C07C 323/62; C07D 277/26

(52) U.S. Cl. 546/283.7; 568/74; 568/77; 568/75; 549/453; 564/440; 562/431; 548/203

(57) ABSTRACT

An alkyl aryl sulfide of Chemical Formula (III) is prepared by substituting an aryl halogen compound of Chemical Formula (I) with an alkyl lithium organometallic reagent. The sulfide is subsequently reacted with a compound of Chemical Formula (II). Alternatively an aryl halogen compound of Chemical Formula (I) is reacted with Grignard reagent to protect the hydrogen-donating substituent, and then reacted with an alkyl lithium organometallic reagent, and subsequently with sulfur and a compound of Chemical Formula (II). An alkyl aryl sulfide of Chemical Formula (III) is prepared via a one-step reaction without separation or purification of an intermediate compound from various aryl halogen compounds.

### FIELD OF THE INVENTION

The present invention relates to a process for preparing sulfide compounds from aryl halogen compounds having various electron-donating, electron-withdrawing or hydrogen-donating substituents via one-step reaction, and the sulfide derivatives prepared therefrom. More specifically, the invention relates to a process for preparing various alkyl aryl sulfides represented by Formula (III) which have important roles in syntheses of organic chemistry and medicinal chemistry, via one-step reaction.

### BACKGROUND OF THE RELATED ART

Alkyl aryl sulfide compounds represented by Formula (III) have very wide utility spectrum in organic chemistry and medicinal chemistry. Thus, a variety of processes for preparing such sulfide compounds have been developed by many researchers. Among the processes, a synthetic process wherein a compound of Formula (I) is reacted with an organic compound containing alkyl or arylthio alcohol and halogen in the presence of a strong base is the most generalized process (Synthesis 1972, 101, 1977, 357, Chemical Reviews 1978, 78, 363). As another synthetic process, there is a carbon-sulfur bonding reaction using palladium (Pd) or copper (Cu) catalyst, as shown in Reaction Scheme (2) (J. Am. Chem. Soc., 1995, 117, 11598, J. Org. Chem. 1998, 63, 9606, 2001, 66, 8677, Aus. J. Chem. 1985, 38, 899, Org. Lett. 2000, 2, 2019, 2002, 4, 2803). In addition, a synthetic process of a sulfide was patented as an important stage in synthesis of GW501516 which has been known as a therapeutic agent for hypertension and hypercholesterolemia, and cardiac disorders caused by such diseases, as shown in Reaction Scheme (3) and (4) (PCT Laid-open Publication WO 01/00603 A1).

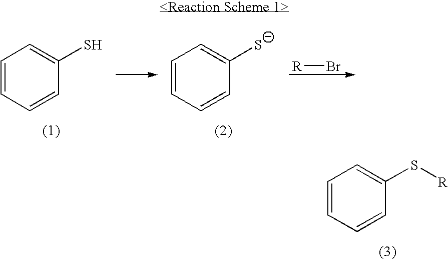

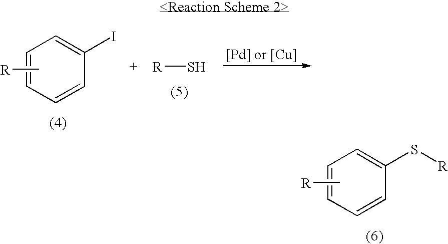

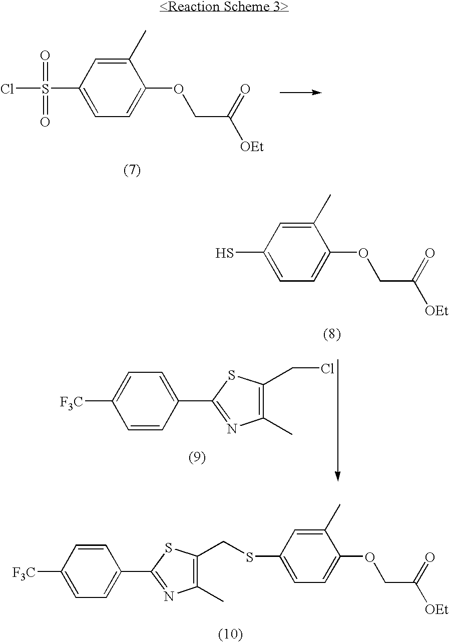

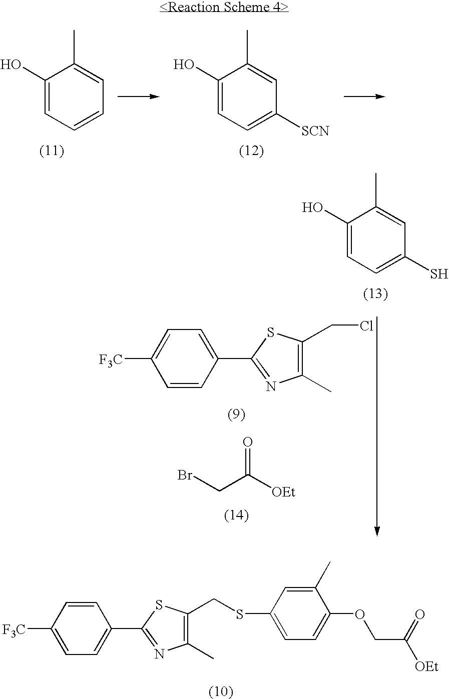

In spite of having a lot of utilities, sulfide derivatives have following deficiencies in their process for preparation:
1) Thiophenol, in the process according to Reaction Scheme (1), has very irritating odor, to cause difficulties in handling. It is unstable in the air and readily forms undesirable aryl disulfides (R—SS—R), having no various kinds commercially available, but rather higher price compared to aryl halogen compounds.
2) The process according to Reaction Scheme (2), though having a high yield (80% or more) of the final product, has severe reaction condition (100° C. or more), with long reaction time of 10 to 24 hours. In addition, because of using unstable thiol compound, it has same deficiencies as in Reaction Scheme (1).
3) According to the process of Reaction Scheme (3), thiol of Chemical Formula (8) is obtained from the compound of Chemical Formula (7) via metallic reduction. However, since the substance represented by Chemical Formula (8) readily forms a disulfide compound to cause problems in storage, it should be reacted with a halogen compound and an inorganic salt immediately after the synthesis. In addition, the process has a limitation as a method to prepare various sulfides, because the position of —SH group introduced by a sulfonyl chloride is essentially para-position.
4) The process for preparing a compound of Chemical Formula (10) in Reaction Scheme (3) is troublesome because a thiol compound (8) must be prepared according to the Scheme and then separated and purified before subsequent reaction.
5) According to the process of Reaction Scheme (4), a thiol of Chemical Formula (13) is prepared from the compound of Chemical Formula (12) via reduction with LiAlH₄. However, this process also has problems in that a disulfide compound is readily formed, and LiAlH₄ employed in the reduction is unstable in the air to give trouble in the production in a large scale.

Thus, a method to rapidly prepare an alkyl aryl sulfide with simple and low-costed process has been required.

### SUMMARY OF THE INVENTION

It is the object of the present invention to provide a process for preparing alkyl aryl sulfides of Chemical Formula (III) via one-step reaction without separation or purification of an intermediate compound, from low-priced and various aryl halogen compounds in a short reaction time with high yield.

As considering the situation described above, the present inventors performed intensive studies on this matter and found that an alkyl aryl sulfide of Chemical Formula (III) is obtained by substituting a halogen from an aryl halogen compound with alkyl lithium organometallic reagent when the compound of Chemical Formula (I) has an electron-donating or an electron-withdrawing substituent, and continuously reacting with the compound of Chemical Formula (II) and sulfur; or by reacting the aryl halogen compound of Chemical Formula (I) containing a hydrogen-donating substituent (—OH, —NH₂, —NRH, —COOH) with an alkylmagnesium halide (Grignard reagent) to protect the hydrogen-donating substituent; substituting the halogen with alkyl lithium organometallic reagent, and continuously reacting with the compound of Chemical Formula (II) and sulfur, to complete the invention.

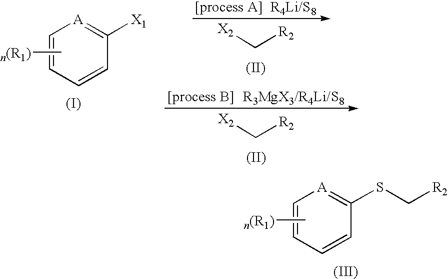

wherein, A represents CH or a nitrogen atom,

X₁ represents a halogen atom,

X₂ represents a halogen atom or a leaving group,

X₃ represents a halogen atom,

R₁ represents a hydrogen atom, a halogen atom, a C₁-C₇ alkyl group, a C₁-C₇ alkyloxy group, a C₁-C₇ alkylthiooxy group, an aryl group, a hydroxyl group, a hydroxymethyl group, a hydroxyethyl group, an amine group, an aminomethyl group, an aminoethyl group, an alkylamine group, a dialkylamine group or a carboxy group, wherein the alkyl group may be substituted by one or more substituent(s) selected from the group consisting of halogen atoms and a hydroxyl group,

R₂ represents a C₁-C₁₀ alkyl group, an aryl group, a C₁-C₁₀ alkylester group, a C₁-C₁₀ alkylketone group or an arylketone group,

R₃ and R₄ independently represents a C₁-C₄ alkyl group, and

n represents an integer of 1 to 3.

Thus, the present invention provides a process for easily and economically preparing various alkyl aryl sulfide derivatives of Chemical Formula (III) by reacting a compound of Chemical Formula (I) with a compound of Chemical Formula (II) via Process (A) or (B) without any separation or purification stage.

Among the compounds represented by Chemical Formula (III) which is prepared via Process (A) or (B) in the Reaction Scheme, novel compounds are 2-(pent-2-ynylsulfanyl)-4-fluorophenol, 2-(5-phenylpentylsulfanyl)-4-fluorophenol, 2-(cyclohexylmethylsulfanyl)-4-fluorophenol, 4-((2-(1,3-dioxolan-2-yl)ethylsulfanyl)phenol, 2-(2-hydroxyhex-5-enylsulfanyl)-4-fluorophenol, 4-((tert-butoxycarbonyl)methylsulfanyl)benzoic acid, 3-(2-(1,3-dioxolan-2-yl)ethylsulfanyl)benzoic acid, 3-(2-hydroxyhex-5-enylsulfanyl)benzoic acid, 2-(4-(benzylsulfanyl)phenyl)ethanol, 2-(3-(benzylsulfanyl)phenyl)ethanol, 1-((4-(hydroxymethyl)phenyl)sulfanyl)hex-5-en-2-ol, (4-(2-(1,3-dioxolan-2-yl)ethylsulfanyl)phenyl)methanol, tert-butyl 2-((4-(hydroxymethyl)phenyl)sulfanyl)acetate, (4-(pent-2-ynylsulfanyl)phenyl)methanol, 4-(benzylthio)-2-bromobenzenamine, 4-(5-phenylpentylthio)benzenamine, 1-(4-aminopentylthio)hex-5-en-2-ol, 2-[4-(benzylthio)phenyl]ethanamine, tert-butyl 2-[4-(2-aminoethyl)phenylthio]-2-methylpropionate, benzyl 2-trifluoromethylphenyl sulfide, benzyl 2-methoxyphenylsulfide, 2-bromo-6-(2-[1,3]dioxolan-2-yl-ethylsulfanyl)pyridine, 5-[4-(tert-butyldimethylsilanyloxy)-3-methylphenylsulfanyl]-4-methyl-2-[(4-trifluoromethyl)phenyl]thiazol, 4-benzylsulfanyl-2-methyl-phenylamine, tert-butyl [4-(2-aminoethyl)phenylthio]acetate, 4-benzylsulfanyl-2,6-dimethylphenol, 4-benzylsulfanyl-2-chlorophenol, 4-benzylsulfanyl-4-fluorophenol, (4-benzylsulfanylphenyl)methanol, tert-butyl(4-hydroxyphenylsulfanyl)acetate, and 2-methyl-4-[[[4-methyl-2-[(4-trifluoromethyl)phenyl]thiazol-5-yl]methyl]sulfanyl]phenol.

Thus the present invention provides useful novel compounds of use.

Other and further objects, features and advantages of the invention will appear more fully from the following description.

### DETAILED DESCRIPTION OF THE PREFERRED EMBODIMENT

In the Reaction Scheme shown above, A represents CH or a nitrogen atom contained in the aryl compound having a resonance structure.

X₁ represents a halogen atom. As the halogen atom, mentioned can be a fluorine atom, chlorine atom, bromine atom, and iodine atom. Among them, a bromine atom or an iodine atom is preferable.

X₂ means a leaving group. A conventional leaving group, specifically a halogen atom, a methansulfonyl oxy group, a p-toluenesulfonyloxy group may be employed. Herein, the halogen atoms include a fluorine atom, a chlorine atom, a bromine atom and an iodine atom. Among them, a halogen atom is preferable, a chlorine atom, a bromine atom, or an iodine atom being more preferable.

X₃, a halogen atom of Grignard reagent, represents a chlorine atom, a bromine atom or an iodine atom.

R₁ represents a hydrogen atom, a halogen atom, a C₁-C₇ alkyl group, a C₁-C₇ alkyloxy group, a C₁-C₇ alkylthiooxy group, an aryl group, a hydroxyl group, a hydroxymethyl group, a hydroxyethyl group, an amine group, an aminomethyl group, an aminoethyl group, an alkylamine group, a dialkylamine group or a carboxy group, where in the alkyl group may be substituted by one or more substituent(s) selected from the group consisting of halogen atoms and a hydroxyl group. Each substituent R₁ may have ortho-, meta- or para-position with respect to the halogen atom (X₁), and number of the substituent(s) is from 1 to 3.

R₂ represents a C₁-C₁₀ alkyl group, an aryl group, a C₁-C₁₀ alkylester group, a C₁-C₁₀ alkylketone group or an arylketone group.

R₃ and R₄ independently represent a C₁-C₄ alkyl group, such as methyl, ethyl, propyl, isopropyl, n-butyl, sec-butyl and tert-butyl group.

In the preparation process according to the present invention, the compound of Chemical Formula (I) employed as raw material is well known in the art and commercially available.

The preparation process according to the invention is now described in detail.

### [Process A] Preparation of an Alkyl Aryl Sulfide Compound Represented by Chemical Formula (III) Having (an) Electron-Donating or Electron-Withdrawing Substituent(s)

An alkyl aryl sulfide compound represented by Chemical Formula (III) is obtained by reacting a compound represented by Chemical Formula (I) with an alkyl lithium organometallic reagent and sulfur, and then with a compound represented by Chemical Formula (II).

Dry solvent such as diethyl ether, tetrahydrofuran, hexane and heptane is used in this process either alone or in a mixture of the two or more. Among them, diethylether, tetrahydrofuran, and a mixture of diethyl ether and tetrahydrofuran are the most preferable.

The alkyl lithium organometallic reagents employed in the halogen-metal substitution include n-butyl lithium, sec-butyl lithium, tert-butyl lithium, and the like. The amount of alkyl lithium organometallic reagent employed is usually from 1 to 3 equivalents with respect to the compound of Chemical Formula (I), most preferably from 1 to 1.2 equivalents in case of n-butyl lithium or sec-butyl lithium, and from 2 to 2.2 equivalents in case of tert-butyl lithium.

Sulfur used in this process is in a powdery state colored pale yellow, and the amount is usually from 1 to 3 equivalents, preferably from 1 to 1.2 equivalents with respect to the compound of Chemical Formula (I).

The reaction temperature varies depending upon the solvent employed, but usually is from −100° C. to 25° C. Preferably the substitution of halogen with metal and introduction of sulfur are carried out at −75° C., and the reaction with compound of Chemical Formula (II) at room temperature (25° C.). The reaction time varies depending on the reaction temperature and the type of solvent employed, but usually is from 30 minutes to 6 hours, preferably 1 hour or less.

### [Process B] Preparation of an Alkyl Aryl Sulfide Compound Represented by Chemical Formula (III) Having a Hydrogen-Donating Substituent

In preparing an alkyl aryl sulfide compound represented by Chemical Formula (III) where the substituent of the compound of Chemical Formula (I) is a hydrogen-donating substituent (—OH, —CH₂OH, —CH₂CH₂OH, —NH₂, —NRH, —CH₂NH₂, —CH₂CH₂NH₂, —COOH), the hydrogen-donating substituent is firstly protected with Grignard reagent, and then reacted with an alkyl lithium organometallic reagent and sulfur, followed by a compound represented by Chemical Formula (II), to obtain a compound of Chemical Formula (III).

As dry solvent used in this process, diethyl ether, tetrahydrofuran, hexane or heptane may be used alone or in a combination of the two or more. Among them, most preferable are diethyl ether, tetrahydrofuran, or a mixture of diethyl ether and tetrahydrofuran.

As the Grignard reagent which protects the hydrogen-donating substituent (—OH, —CH₂OH, —CH₂CH₂OH, —NH₂, —NRH, —CH₂NH₂, —CH₂CH₂NH₂, —COOH), employed may be CH₃MgCl, CH₃MgBr, CH₃MgI, CH₃CH₂MgCl, CH₃CH₂MgBr, CH₃CH₂MgI, CH₃CH₂CH₂MgCl, CH₃CH₂CH₂MgBr, CH₃CH₂CH₂MgI, (CH₃)₂CHMgCl, (CH₃)₂CHMgBr, (CH₃)₂CHMgI, CH₃CH₂CH₂CH₂MgCl, CH₃CH₂CH₂CH₂MgBr, CH₃CH₂CH₂CH₂MgI, C₂H₅CHCH₃MgCl, C₂H₅CHCH₃MgBr, C₂H₅CHCH₃MgI, (CH₃)₃CMgCl, (CH₃)₃CMgBr, (CH₃)₃CMgI, or the like. Among them, R₃MgCl and R₃MgBr are preferable, (CH₃)₂CHMgC₁ and CH₃CH₂CH₂CH₂MgCl being more preferable.

The alkyl lithium organometallic reagents which can be employed in halogen-metal substitution reaction include n-butyl lithium, sec-butyl lithium, tert-butyl lithium, and the like. The amount of alkyl lithium organometallic reagent employed is usually from 1 to 3 equivalents, more preferably from 1 to 1.2 equivalents in case of n-butyl lithium or sec-butyl lithium, from 2 to 2.2 equivalents in case of tert-butyl lithium.

Sulfur used in this process is in a powdery state colored pale yellow, and the amount is usually from 1 to 3 equivalents, preferably from 1 to 1.2 equivalents with respect to the compound of Chemical Formula (I).

The reaction temperature varies depending upon the solvent employed, but usually is from −100° C. to 25° C. Preferably, protection of the hydrogen-donating substituent is carried out at 0° C. to 25° C., the substitution of halogen with metal and introduction of sulfur at −75° C., and the reaction with compound of Chemical Formula (II) at room temperature (25° C.). The reaction time varies depending on the reaction temperature and the type of solvent employed, but usually is from 30 minutes to 6 hours, preferably 2 hours or less.

The present invention regarding alkyl aryl sulfide compounds of Chemical Formula (III) thus obtained and processes for preparing the same are very important in production process of primary intermediates in organochemical reactions and therapeutic agents containing alkyl aryl sulfide functional groups among therapeutic agents for treating various diseases.

### EXAMPLES

The present invention is described specifically below by way of Examples. However, the present invention is not restricted to these Examples.

### Example 1

### Preparation of benzyl 2-trifluoromethylphenyl sulfide

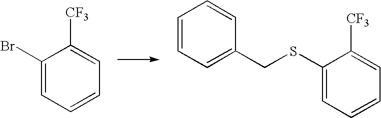

1-Bromo-2-(trifluoromethyl)-benzene 271 μl (2 mmol) was completely dissolved in dry tetrahydrofuran 15 ml under nitrogen atmosphere, and the mixture was cooled to −78° C. To the mixture, n-butyl lithium 1.25 ml (1.6M in hexane, 1.0 equivalent) was slowly added for 1 minute. After stirring additional 10 minutes, sulfur powder 64 mg (2 mmol, 1.0 equivalent) was added at once at the same temperature. After stirring the mixture at the same temperature for additional 10 minutes to completely dissolve sulfur, benzyl bromide 236 μl (2 mmol, 1.0 equivalent) was slowly added thereto. Reaction was carried out so that the temperature of overall reaction mixture was raised to room temperature in 20 minutes. The reaction was monitored by TLC, and when the reaction was completed, 15 ml of aqueous ammonium chloride solution was added thereto to quench the reaction. The organic phase was extracted by using ethyl acetate. Moisture contained in the organic phase was removed by using magnesium sulfate. After filtration, the solvent was evaporated under reduced pressure. The residue was purified by silica gel column chromatography to obtain the title compound 440 mg (yield: 82%).

¹H-NMR (300 MHz, CDCl₃) δ: 7.63 (d, 1H, J=7.6 Hz), 7.38 (d, 2H, J=3.4 Hz), 7.32˜7.23 (m, 6H), 4.15 (s, 2H).

¹³C-NMR (75.5 MHz, CDCl₃) δ: 136.8, 136.2, 132.3, 132.2, 129.9, 129.4, 128.9, 127.2 (q, J=3.7 Hz), 123.3, 39.7.

### Example 2

### Preparation of benzyl 3-trifluoromethylphenyl sulfide

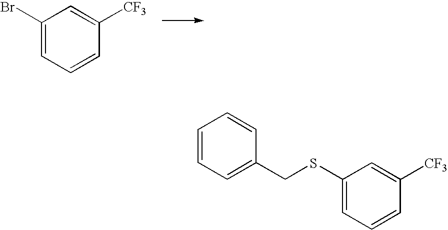

The same procedure as described in Example 1 was repeated but 1-bromo-2-(trifluoromethyl)-benzene was replaced with 1-bromo-3-(trifluoromethyl)-benzene 276 μl (2 mmol). After purification, the title compound 381 mg (yield: 71%) was obtained.

¹H-NMR (300 MHz, CDCl₃) δ: 7.51 (br s, 1H), 7.39 (t, 2H), 7.33 (d, 1H), 7.28 (m, 5H), 4.13 (s, 2H).

¹³C-NMR (75.5 MHz, CDCl₃) δ: 138.3, 137.0, 133.0, 131.6 (q, J=32 Hz), 129.5, 129.2, 129.0, 128.9, 127.9, 126.5 (q, J=3.7 Hz), 123.3, 39.1.

### Example 3

### Preparation of benzyl 4-trifluoromethylphenyl sulfide

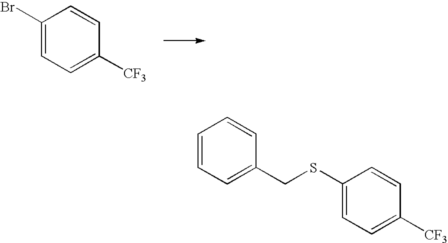

The same procedure as described in Example 1 was repeated but 1-bromo-2-(trifluoromethyl)-benzene was replaced with 1-bromo-4-(trifluoromethyl)-benzene 276 μl (2 mmol). After purification, the title compound 515 mg (yield: 96%) was obtained.

¹H-NMR (300 MHz, CDCl₃) δ: 7.48 (d, 2H, J=8.2 Hz), 7.36˜7.25 (m, 7H), 4.19 (s, 2H).

¹³C-NMR (75.5 MHz, CDCl₃) δ: 142.5, 136.7, 129.1, 129.0, 128.3, 127.9, 126.0 (q, J=3.9 Hz), 38.1.

### Example 4

### Preparation of benzyl 2-methoxyphenyl sulfide

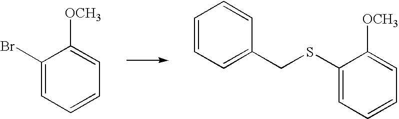

The same procedure as described in Example 1 was repeated but 1-bromo-2-(trifluoromethyl)-benzene was replaced with 2-bromoanizol 248 μl (2 mmol). After purification, the title compound 350 mg (yield: 76%) was obtained.

¹H-NMR (300 MHz, CDCl₃) δ: 7.30˜7.18 (m, 7H), 6.84 (m, 2H, J=7.8 Hz), 4.09 (s, 2H), 3.88 (s, 3H).

¹³C-NMR (75.5 MHz, CDCl₃) δ: 157.9, 137.9, 130.8, 129.3, 128.8, 128.0, 127.4, 124.8, 121.4, 110.9, 56.2, 37.7.

### Example 5

### Preparation of benzyl 3-methoxyphenyl sulfide

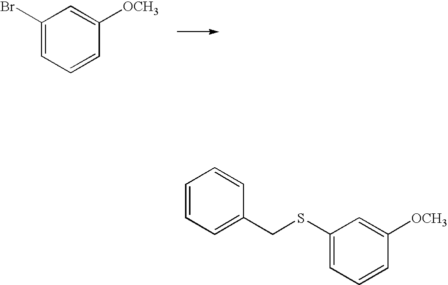

The same procedure as described in Example 1 was repeated but 1-bromo-2-(trifluoromethyl)-benzene was replaced with 3-bromoanizol 251 μl (2 mmol). After purification, the title compound 332 mg (yield: 72%) was obtained.

¹H-NMR (300 MHz, CDCl₃) δ: 7.32˜7.24 (m, 5H), 7.16 (t, 1H), 6.86 (d, 1H), 6.82 (t, 1H), 6.65 (dd, 1H), 4.11 (s, 2H), 3.73 (s, 3H).

¹³C-NMR (75.5 MHz, CDCl₃) δ: 160.1, 138.1, 137.8, 130.0, 129.2, 128.9, 127.6, 122.1, 115.2, 112.6, 55.6, 39.2.

### Example 6

### Preparation of benzyl 4-methoxyphenyl sulfide

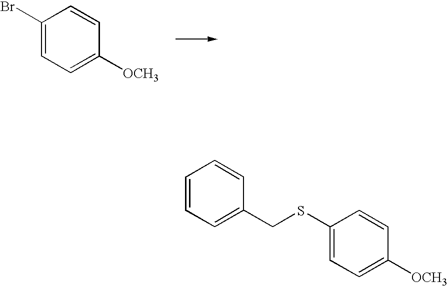

The same procedure as described in Example 1 was repeated but 1-bromo-2-(trifluoromethyl)-benzene was replaced with 4-bromoanizol 250 μl (2 mmol). After purification, the title compound 424 mg (yield: 92%) was obtained.

¹H-NMR (300 MHz, CDCl₃) δ: 7.28˜7.16 (m, 7H), 6.77 (d, 2H, J=8.6 Hz), 3.97 (s, 2H), 3.76 (s, 3H).

¹³C-NMR (75.5 MHz, CDCl₃) δ: 159.6, 138.5, 134.5, 129.8, 129.3, 128.8, 127.3, 114.8, 55.7, 41.6.

### Example 7

### Preparation of benzyl 4-biphenyl sulfide

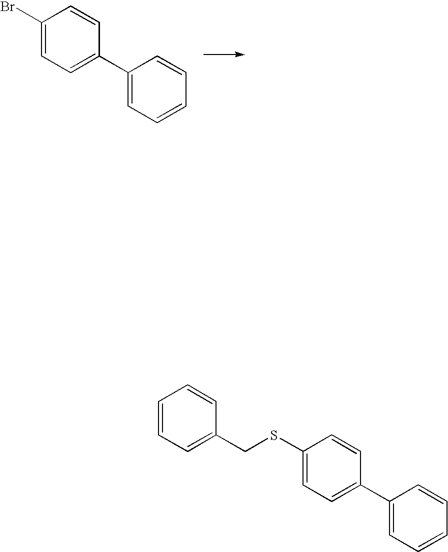

The same procedure as described in Example 1 was repeated but 1-bromo-2-(trifluoromethyl)-benzene was replaced with 4-bromobiphenyl 468 μl (2 mmol). After purification, the title compound 514 mg (yield: 93%) was obtained.

¹H-NMR (300 MHz, CDCl₃) δ: 7.70˜7.20 (m, 14H), 4.15 (s, 2H).

¹³C-NMR (75.5 MHz, CDCl₃) δ: 140.8, 139.6, 137.8, 135.9, 131.8, 130.4, 129.3, 129.2, 128.9, 128.3, 127.9, 127.7, 127.6, 127.4, 127.3, 39.5.

### Example 8

### Preparation of tert-butyl 4-methoxyphenylsulfanylacetate

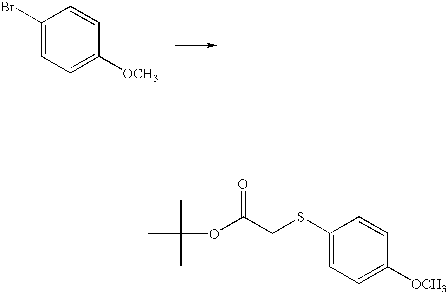

4-bromoanizol 374 mg (2 mmol) was completely dissolved in dry tetrahydrofuran 15 ml under nitrogen atmosphere, and the mixture was cooled to −78° C. To the mixture, n-butyl lithium 1.25 ml (1.6M in hexane, 1.0 equivalent) was slowly added for 1 minute. After stirring additional 10 minutes, sulfur powder 64 mg (2 mmol, 1.0 equivalent) was added at once at the same temperature. After stirring the mixture at the same temperature for additional 5 minutes to completely dissolve sulfur, tert-butyl bromoacetate 295 μl (2 mmol, 1.0 equivalent) was slowly added. Reaction was carried out so that the temperature of overall reaction mixture was raised to room temperature in 20 minutes. When the reaction was completed, aqueous ammonium chloride solution was added thereto to quench the reaction. The organic phase was extracted by using ethyl acetate and aqueous sodium chloride solution. Moisture contained in the organic phase was removed by using magnesium sulfate. After filtration, the solvent was evaporated under reduced pressure. The residue was purified by silica gel column chromatography to obtain the title compound 488 mg (yield: 96%).

¹H-NMR (300 MHz, CDCl₃) δ: 7.42 (d, 2H, J=8.8 Hz), 6.83 (d, 2H, J=8.8 Hz), 3.79 (s, 3H), 3.43 (s, 2H), 1.39 (s, 9H).

¹³C-NMR (75.5 MHz, CDCl₃) δ: 169.4, 159.9, 134.3, 125.7, 114.9, 81.9, 55.7, 40.0, 28.3.

### Example 9

### Preparation of 1-allyl 4-methoxyphenyl sulfide

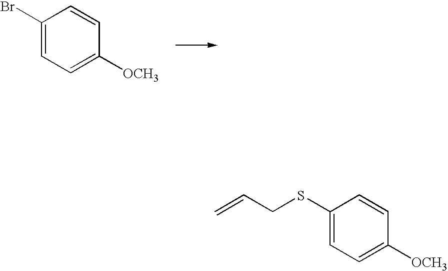

The same procedure as described in Example 8 was repeated but tert-butyl bromoacetate was replaced with allylbromide 173 μl (2 mmol). After purification, the title compound 328 mg (yield: 91%) was obtained.

¹H-NMR (300 MHz, CDCl₃) δ: 7.33 (d, 2H, J=9.8 Hz), 6.82 (d, 2H, J=9.8 Hz), 5.82 (m, 1H), 5.01 (s, 1H), 4.97 (dd, 1H, J=8.0 and 1.3 Hz), 3.78 (s, 3H), 3.42 (d, 2H, J=7 Hz).

¹³C-NMR (75.5 MHz, CDCl₃) δ: 159.5, 134.4, 134.3, 126.2, 117.6, 114.8, 55.7, 39.7, 30.7.

### Example 10

### Preparation of 1-butyl-4-methoxyphenyl sulfide

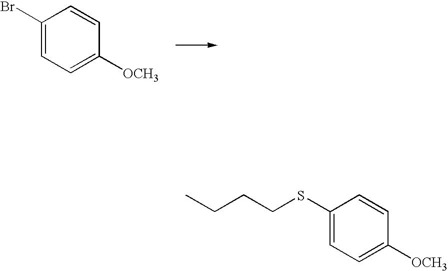

The same procedure as described in Example 8 was repeated but using reaction intermediate, 1-bromobutane, instead of using additional butylhalide. After purification, the title compound 295 mg (yield: 75%) was obtained.

¹H-NMR (300 MHz, CDCl₃) δ: 7.32 (d, 2H, J=8.8 Hz), 6.83 (d, 2H, J=8.7 Hz), 3.79 (s, 3H), 2.82 (t, 2H), 1.55 (m, 2H), 1.40 (m, 2H), 0.89 (t, 3H).

¹³C-NMR (75.5 MHz, CDCl₃) δ: 159.1, 133.3, 127.3, 114.9, 55.7, 35.9, 31.8, 22.2, 14.0.

### Example 11

### Preparation of 1-(4-methoxyphenylsulfanyl)-propan-2-one

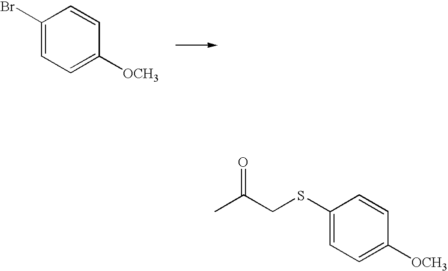

The same procedure as described in Example 8 was repeated but tert-butyl bromoacetate was replaced with chloroacetone 159 μl (2 mmol). After purification, the title compound 357 mg (yield: 91%) was obtained.

¹H-NMR (300 MHz, CDCl₃) δ: 7.35 (d, 2H, J=8.8 Hz), 6.83 (d, 2H, J=8.8 Hz), 3.79 (s, 3H), 3.54 (s, 2H), 2.26 (s, 3H).

¹³C-NMR (75.5 MHz, CDCl₃) δ: 204.0, 160.0, 134.0, 125.0, 115.2, 55.7, 46.9, 28.4.

### Example 12

### Preparation of 2-(4-methoxyphenylsulfanyl)-1-phenylethanone

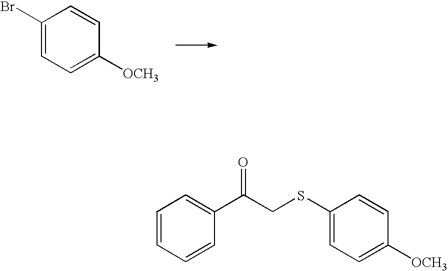

The same procedure as described in Example 8 was repeated but tert-butyl bromoacetate was replaced with 2-bromoacetophenone 398 mg (2 mmol). After purification, the title compound 486 mg (yield: 94%) was obtained.

¹H-NMR (300 MHz, CDCl₃) δ: 7.90 (d, 2H, J=7.2 Hz), 7.55 (t, 1H), 7.43 (t, 2H), 7.34 (d, 2H, J=8.8 Hz), 6.80 (d, 2H, J=6.8 Hz), 4.12 (s, 2H), 3.76 (s, 3H).

¹³C-NMR (75.5 MHz, CDCl₃) δ: 194.7, 160.1, 135.9, 135.0, 133.7, 129.1, 129.0, 125.0, 115.1, 55.7, 43.2.

### Example 13

### Preparation of 2-bromo-6-(2 [1,3]dioxolan-2-yl-ethylsulfanyl)pyridine

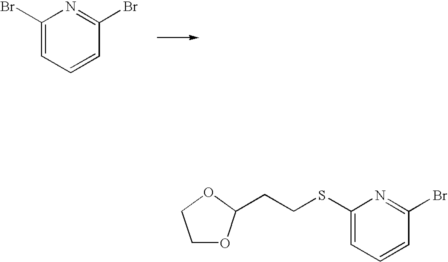

2,6-Dibromo pyridine 476 mg (2 mmol) was completely dissolved in dry tetrahydrofuran 15 ml under nitrogen atmosphere, and the mixture was cooled to −78° C. To the mixture, butyl lithium 1.25 ml (1.6M in hexane, 1.0 equivalent) was slowly added for 1 minute. After stirring additional 10 minutes, sulfur powder 64 mg (2 mmol, 1.0 equivalent) was added at once at the same temperature. After stirring the mixture at the same temperature for additional 5 minutes to completely dissolve sulfur powder, 2-(2bromoethyl)-1,3-dioxolan 261 μl (2 mmol, 1.0 equivalent) was slowly added. Reaction was carried out so that the temperature of overall reaction mixture was raised to room temperature in 20 minutes. when the reaction was completed, aqueous ammonium chloride solution was added thereto to quench the reaction. The organic phase was extracted by using ethyl acetate and aqueous sodium chloride solution. Moisture contained in the organic phase was removed by using magnesium sulfate. After filtration, the solvent was evaporated under reduced pressure. The residue was purified by silica gel column chromatography to obtain the title compound 459 mg (yield: 79%).

¹H-NMR (300 MHz, CDCl₃) δ: 7.35˜7.11 (m, 3H), 5.05 (t, 1H), 4.02 (m, 2H), 3.91 (m, 2H), 3.27 (t, 2H), 2.11 (m, 2H)

¹³C-NMR (75.5 MHz, CDCl₃) δ: 160.7, 142.0, 138.3, 123.5, 121.0, 103.6, 65.3, 33.7, 25.3.

### Example 14

### Preparation of 4-(4-bromobenzylsulfanyl)benzoic acid

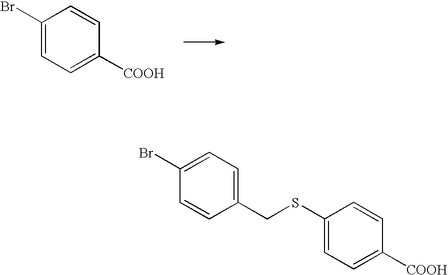

4-bromobenzoic acid 402 mg (2 mmol) was completely dissolved in dry tetrahydrofuran 15 ml under nitrogen atmosphere, and the mixture was cooled to −78° C. To the mixture, butyl lithium 2.5 ml (1.6M in hexane, 1.0 equivalent) was slowly added for 1 minute. After stirring additional 10 minutes, sulfur powder 64 mg (2 mmol, 1.0 equivalent) was added at once at the same temperature. After stirring the mixture at the same temperature for additional 5 minutes to completely dissolve sulfur powder, 4-bromobenzylbromide 250 mg (2 mmol, 1.0 equivalent) was slowly added. Reaction was carried out so that the temperature of overall reaction mixture was raised to room temperature in 20 minutes. when the reaction was completed, aqueous ammonium chloride solution was added thereto to quench the reaction. The organic phase was extracted by using ethyl acetate and aqueous sodium chloride solution. Moisture contained in the organic phase was removed by using magnesium sulfate. After filtration, the solvent was evaporated under reduced pressure. The residue was purified by silica gel column chromatography to obtain the title compound 575 mg (yield: 89%).

¹H-NMR (300 MHz, DMSO-d₆) δ: 12.9 (br s, 1H), 7.82 (d, 2H, J=8.4 Hz), 7.50 (d, 2H, J=8.4 Hz), 7.40 (d, 2H, J=11.8 Hz), 7.37 (d, 2H, J=11.8 Hz), 4.33 (s, 2H).

¹³C-NMR (75.5 MHz, DMSO-d₆) δ: 167.7, 143.3, 137.4, 132.2, 131.8, 130.5, 128.4, 127.5, 121.1, 35.4.

### Example 15

### Preparation of 2-(benzylsulfanyl)benzoic acid

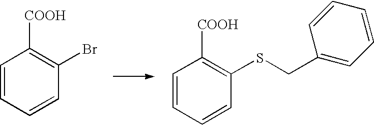

2-bromobenzoic acid 402 mg (2 mmol) was completely dissolved in dry tetrahydrofuran 15 ml under nitrogen atmosphere, and the mixture was cooled to 0° C. To the mixture, isopropylmagnesium chloride 1.0 ml (2.0 mmol, 2.0 M-ether, 1.0 equivalent) was slowly added at the same temperature. After 10 minutes, the mixture was cooled to −78° C. tert-Butyl lithium 2.35 μl (4.0 mmol, 1.7 M-pentane, 2.0 equivalent) was slowly added for 1 minute. Sulfur powder 64 mg (2 mmol) dissolved in dry tetrahydrofuran 3.0 μl was added. Reaction was carried out so that the temperature of overall reaction mixture was raised to room temperature in 30 minutes. Again, the mixture was cooled to 0° C. Benzylbromide 238 μl (2 mmol, 1.0 equivalent) was slowly added to the mixture. After 20 minutes at room temperature, aqueous ammonium chloride solution was added thereto to quench the reaction. The organic phase was extracted by using ethyl acetate and 5% aqueous hydrochloric acid solution. Moisture contained in the organic phase was removed by using magnesium sulfate. After filtration, the solvent was evaporated under reduced pressure. The residue was purified by silica gel column chromatography to obtain the title compound 429 mg (yield: 88%).

¹H-NMR (300 MHz, DMSO-d₆) δ: 13.00 (s, 1H) 7.89-7.86 (m, 1H), 7.49-7.20 (m, 8H), 4.19 (s, 2H).

¹³C-NMR (75 MHz, DMSO-d₆) δ: 168.27, 142.07, 137.49, 133.18, 131.77, 130.02, 129.33, 128.58, 128.01, 126.59, 124.85, 36.57

### Example 16

### Preparation of 3-(benzylsulfanyl)benzoic acid

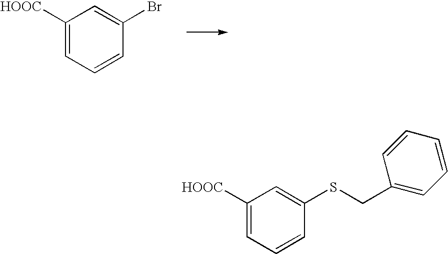

The same procedure as described in Example 15 was repeated but 2-bromobenzoic acid was replaced with 3-bromobenzoic acid 402 mg (2 mmol). After purification, the title compound 473 mg (yield: 97%) was obtained.

¹H-NMR (300 MHz, DMSO-d₆) δ: 13.04 (s, 1H), 8.12-7.21 (m, 9H), 4.28 (s, 2H).

¹³C-NMR (75 MHz, DMSO-d₆) δ: 167.63, 137.96, 137.79, 133.22, 132.34, 130.11, 130.06, 129.69, 129.48, 129.41, 129.26, 128.01, 127.54, 37.36.

### Example 17

### Preparation of 4-(benzylsulfanyl)benzoic acid

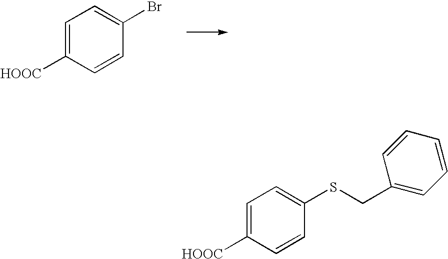

The same procedure as described in Example 15 was repeated but 2-bromobenzoic acid was replaced with 4-bromobenzoic acid 402 mg (2 mmol). After purification, the title compound 449 mg (yield: 92%) was obtained.

¹H-NMR (300 MHz, DMSO-d₆) δ: 12.85 (s, 1H), 7.82-7.79 (m, 2H), 7.41-7.20 (m, 7H), 4.33 (s, 2H).

¹³C-NMR (75 MHz, DMSO-d₆) δ: 167.77, 143.90, 137.64, 130.56, 129.71, 129.33, 128.29, 128.09, 127.30, 36.2.

### Example 18

### Preparation of 4-((tert-butoxycarbonyl)methylsulfanyl)benzoic acid

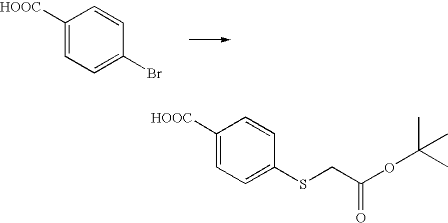

The same procedure as described in Example 15 was repeated but 2-bromobenzoic acid and benzylbromide were replaced with 4-bromobenzoic acid 402 mg (2 mmol) and tert-butyl bromoacetate 295 μl (2 mmol, 1.0 equivalent), respectively. After purification, the title compound 472 mg (yield: 88%) was obtained.

¹H-NMR (300 MHz, DMSO-d) δ: 12.40 (br, 1H), 8.02-7.95 (m, 2H), 7.40-7.37 (m, 2H), 3.60 (s, 2H), 1.43 (s, 9H)

¹³C-NMR (75 MHz, DMSO-d) δ: 172.28, 168.54, 144.25, 130.95, 127.21, 126.89, 82.97, 36.30, 28.

### Example 19

### Preparation of 3-(2-(1,3-dioxolan-2-yl-ethylsulfanyl)benzoic acid

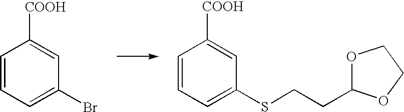

The same procedure as described in Example 15 was repeated but 2-bromobenzoic acid and benzylbromide were replaced with 3-bromobenzoic acid 402 mg (2 mmol) and 2-(2-bromomethyl)-1,3-dioxolan 235 μl (2 mmol, 1.0 equivalent), respectively. After purification, the title compound 416 mg (yield: 82%) was obtained.

¹H-NMR (300 MHz, DMSO-d₆) δ: 11.95 (br, 1H), 8.07-8.06 (m, 1H), 7.92-7.89 (m, 1H), 7.57-7.54 (m, 1H), 7.41-7.35 (m, 1H), 5.03-5.00 (m, 1H), 4.02-3.85 (m, 4H) 3.11-3.06 (m, 2H), 2.07-2.00 (m, 2H).

¹³C-NMR (75 MHz, DMSO-d₆) δ: 172.01, 137.99, 134.14, 130.60, 130.51, 129.37, 127.90, 103.32, 65.41, 33.70, 27.

### Example 20

### Preparation of 3-(2-hydroxyhex-5-enylsulfanyl)benzoic acid

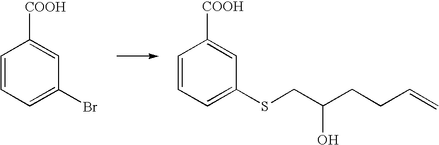

The same procedure as described in Example 15 was repeated but 2-bromobenzoic acid and benzylbromide were replaced with 3-bromobenzoic acid 402 mg (2 mmol) and 1.2-epoxy-5-hexene 228 μl (2 mmol, 1.0 equivalent), respectively. After purification, the title compound 418 mg (yield: 83%) was obtained.

¹H-NMR (300 MHz, DMSO-d₆) δ: 8.09-8.10 (m, 1H), 7.92-7.94 (m, 1H), 7.58-7.62 (m, 1H), 7.37-7.42 (m, 1H), 5.76-5.82 (m, 1H). 4.95-5.07 (m, 2H), 3.75 (m, 1H), 3.18-3.24 (m, 1H), 2.91-2.99 (m, 1H), 2.17-2.24 (m, 2H), 1.63-1.70 (m, 1H).

¹³C-NMR (75 MHz, DMSO-d₆) δ: 171.12, 138.27, 137.10, 135.00, 131.16, 130.62, 129.54, 128.50, 115.59, 69.50, 42.09, 35.58, 30.29.

### Example 21

### Preparation of benzyl 4-bromophenylsulfide

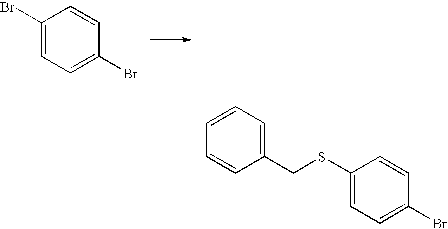

1,4-Dibromo benzene 256 μl (2 mmol) was completely dissolved in dry tetrahydrofuran 15 ml under nitrogen atmosphere, and the mixture was cooled to −78° C. To the mixture, butyl lithium 1.25 ml (1.6M in hexane, 1.0 equivalent) was slowly added for 1 minute. After stirring additional 10 minutes, sulfur powder 64 mg (2 mmol, 1.0 equivalent) was added at once at the same temperature. After stirring the mixture at the same temperature for additional 5 minutes to completely dissolve sulfur, benzyl bromide 236 μl (2 mmol, 1.0 equivalent) was slowly added. Reaction was carried out so that the temperature of overall reaction mixture was raised to room temperature in 20 minutes. When the reaction was completed, aqueous ammonium chloride solution was added thereto to quench the reaction. The organic phase was extracted by using ethyl acetate and aqueous sodium chloride solution. Moisture contained in the organic phase was removed by using magnesium sulfate. After filtration, the solvent was evaporated under reduced pressure. The residue was purified by silica gel column chromatography to obtain the title compound 491 mg (yield: 88%).

¹H-NMR (300 MHz, CDCl₃) δ: 7.36 (d, 2H, J=8.6 Hz), 7.28 (m, 5H), 7.14 (d, 2H, J=8.6 Hz), 4.08 (s, 2H).

¹³C-NMR (75.5 MHz, CDCl₃) δ: 137.4, 135.8, 132.2, 131.9, 128.9, 127.7, 120.7, 39.5.

### Example 22

### Preparation of 1-phenyl-2-(2,4,6-trimethyl phenyl sulfanyl)ethanone

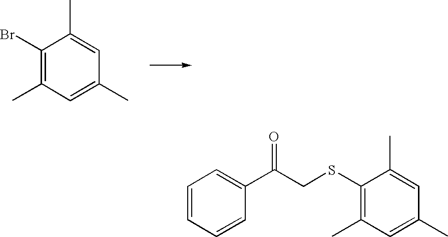

mesityl bromide 300 μl (2 mmol) was completely dissolved in dry tetrahydrofuran 15 ml under nitrogen atmosphere, and the mixture was cooled to −78° C. To the mixture, butyl lithium 1.25 ml (1.6M in hexane, 1.0 equivalent) was slowly added for 1 minute. After stirring additional 10 minutes, sulfur powder 64 mg (2 mmol, 1.0 equivalent) was added at once at the same temperature. After stirring the mixture at the same temperature for additional 5 minutes to completely dissolve sulfur, phenacyl bromide 398 mg (2 mmol, 1.0 equivalent) was slowly added. Reaction was carried out so that the temperature of overall reaction mixture was raised to room temperature in 20 minutes. when the reaction was completed, aqueous ammonium chloride solution was added thereto to quench the reaction. The organic phase was extracted by using ethyl acetate and aqueous sodium chloride solution. Moisture contained in the organic phase was removed by using magnesium sulfate. After filtration, the solvent was evaporated under reduced pressure. The residue was purified by silica gel column chromatography to obtain the title compound 498 mg (yield: 92%).

¹H-NMR (300 MHz, CDCl₃) δ: 7.87 (d, 2H, J=6.3 Hz), 7.55 (t, 1H, J=7.4 Hz), 7.42 (t. 2H, J=6.6 Hz), 6.89 (s, 2H) 3.92 (s, 2H), 2.38 (s, 6H), 2.25 (s, 3H).

¹³C-NMR (75.5 MHz, CDCl₃) δ: 194.9, 143.5, 142.2, 139.2, 135.9, 133.6, 129.5, 129.1, 128.9, 41.2, 22.1, 21.4.

### Example 23

### Preparation of 2-benzylsulfanylphenylamine

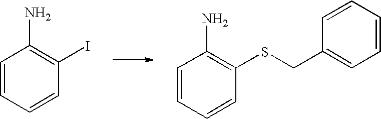

2-iodoaniline 438 mg (2 mmol) was completely dissolved in dry tetrahydrofuran 15 ml under nitrogen atmosphere. To the mixture, isopropylmagnesium chloride 2.0 ml (2.0 M-ether, 2.0 equivalent) was slowly added at 0° C. After 10 minutes at room temperature, the mixture was cooled to −78° C. tert-Butyl lithium 1.18 ml (1.7 M-pentane, 2.0 equivalent) was slowly added for 1 minute. After stirring additional 10 minutes, sulfur powder 64 mg (2 mmol, 1.0 equivalent) was added at once at the same temperature. Reaction was carried out so that the temperature of overall reaction mixture was raised to room temperature in 25 minutes. Again, the mixture was cooled to 0° C. Benzylbromide 236 μl (2 mmol, 1.0 equivalent) was slowly added to the mixture. After 20 minutes at room temperature, when the reaction was completed, aqueous ammonium chloride solution was added thereto to quench the reaction. The organic phase was extracted by using ethyl acetate and aqueous sodium chloride solution. After filtration, the solvent was evaporated under reduced pressure. The residue was purified by silica gel column chromatography to obtain the title compound 254 mg (yield: 59%).

¹H-NMR (300 MHz, CDCl₃) δ: 7.23˜7.12 (m, 7H), 6.68 (d, 1H, J=9.2 Hz), 6.58 (t, 1H, J=17.4 and 8.8 Hz), 4.23 (br s, 2H), 3.89 (s, 2H).

¹³C-NMR (75.5 MHz, CDCl₃) δ: 148.8, 138.5, 136.7, 130.2, 129.1, 128.6, 127.2, 118.7, 115.1, 39.8.

### Example 24

### Preparation of 3-benzylsulfanylphenylamine

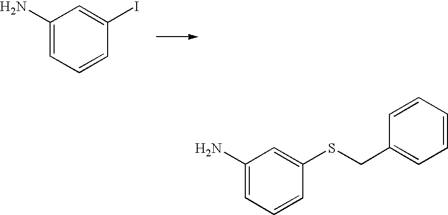

The same procedure as described in Example 23 was repeated but 2-iodoaniline was replaced with 3-iodoaniline 241 μl (2 mmol). After purification, the title compound 284 mg (yield: 66%) was obtained.

¹H-NMR (300 MHz, CDCl₃) δ: 7.29 (m, 5H), 7.03 (t, 1H, J=15.7 and 7.9 Hz), 7.55 (d, 1H, J=8.5 Hz), 6.61 (t, 1H, J=2 Hz), 6.45 (dd, 1H, J=8.8 and 1.8 Hz), 4.09 (s, 2H), 3.60 (br s, 2H).

¹³C-NMR (75.5 MHz, CDCl₃) δ: 146.9, 137.7, 137.6, 129.8, 129.0, 128.7, 127.3, 119.7, 115.9, 113.4, 38.9.

### Example 25

### Preparation of 3-benzylsulfanylphenylamine

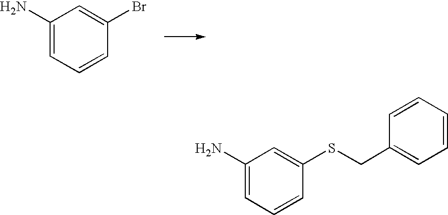

The same procedure as described in Example 23 was repeated but 2-iodoaniline was replaced with 3-bromoaniline 218 μl (2 mmol). After purification, the title compound 198 mg (yield: 46%) was obtained.

¹H-NMR (300 MHz, CDCl₃) δ: 7.29 (m, 5H), 7.03 (t, 1H, J=15.7 and 7.9 Hz), 7.55 (d, 1H, J=8.5 Hz), 6.61 (t, 1H, J=2 Hz), 6.45 (dd, 1H, J=8.8 and 1.8 Hz), 4.09 (s, 2H) 3.60 (br s, 2H).

¹³C-NMR (75.5 MHz, CDCl₃) δ: 146.9, 137.7, 137.6, 129.8, 129.0, 128.7, 127.3, 119.7, 115.9, 113.4, 38.9.

### Example 26

### Preparation of 4-benzylsulfanylphenylamine

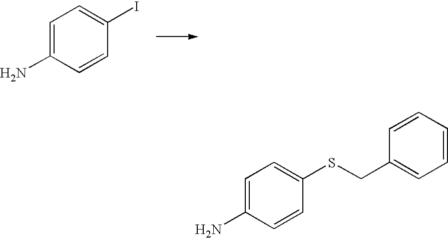

The same procedure as described in Example 23 was repeated but 2-iodoaniline was replaced with 4-iodoaniline 438 mg (2 mmol). After purification, the title compound 396 mg (yield: 92%) was obtained.

¹H-NMR (300 MHz, CDCl₃) δ: 7.21 (m, 5H), 7.11 (d, 2H, J=8.6 Hz), 6.54 (d, 2H, J=8.6 Hz), 3.92 (s, 2H), 3.66 (br s, 2H).

¹³C-NMR (75.5 MHz, CDCl₃) δ: 146.4, 138.6, 134.9, 129.1, 128.5, 127.0, 123.1, 115.6, 41.9.

### Example 27

### Preparation of 4-benzylsulfanyl-2-methyl-phenylamine

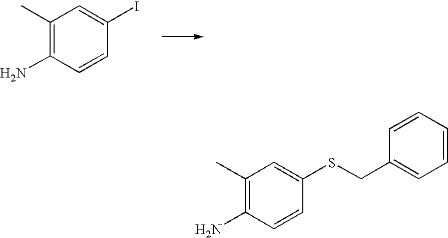

The same procedure as described in Example 23 was repeated but 2-iodoaniline was replaced with 4-iodo-2-methylaniline 466 mg (2 mmol). After purification, the title compound 381 mg (yield: 83%) was obtained.

¹H-NMR (300 MHz, CDCl₃) δ: 7.23 (m, 5H), 7.03 (s, 1H), 7.01 (d, 1H, J=8.1 Hz), 6.53 (d, 1H, J=8.1 Hz), 3.93 (s, 2H), 3.61 (br s, 2H), 2.07 (s, 3H).

¹³C-NMR (75.5 MHz, CDCl₃) δ: 144.6, 138.7, 135.7, 132.3, 129.1, 128.4, 127.0, 123.1, 122.9, 115.4, 41.9, 17.3.

### Example 28

### Preparation of 4-(benzylthio)-2-bromobenzenamine

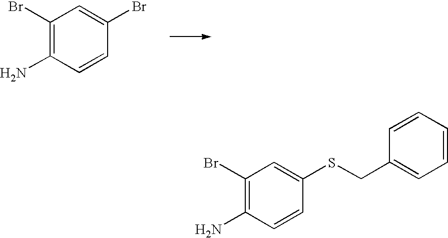

The same procedure as described in Example 23 was repeated but 2-iodoaniline was replaced with 2,4-dibromobenzenamine 502 mg (2 mmol). After purification, the title compound 435 mg (yield: 74%) was obtained.

¹H-NMR (300 MHz, CDCl₃) δ 7.39 (d, 1H), 7.29˜7.12 (m, 5H), 7.04 (dd, 1H), 6.61 (d, 1H), 4.11 (bs, 2H), 3.93 (s, 2H).

¹³C-NMR (75.5 MHz, CDCl₃) δ 144.2, 138.4, 137.6, 134.0, 129.4, 128.8, 127.5, 124.5, 116.0, 109.3, 42.1.

### Example 29

### Preparation of 4-[(4-aminophenylthio)methyl]benzonitrile

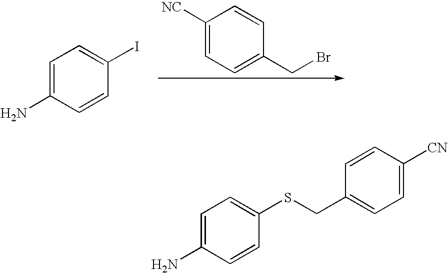

The same procedure as described in Example 23 was repeated but 2-iodoaniline and benzylbromide were replaced with 4-iodobenzenamine 438 mg (2 mmol) and 4-(bromomethyl)benzonitrile 392 mg (2 mmol), respectively. After purification, the title compound 428 mg (yield: 89%) was obtained.

¹H-NMR (300 MHz, CDCl₃) δ 7.50 (dt, 2H), 7.19 (d, 2H), 7.05 (dt, 2H), 6.54 (dt, 2H), 3.89 (s, 2H), 3.75 (bs, 2H).

¹³C-NMR (75.5 MHz, CDCl₃) δ 147.0, 144.5, 135.6, 132.2, 129.8, 121.3, 119.1, 115.6, 110.7, 41.8.

### Example 30

### Preparation of 4-(2-methylallylthio)benzenamine

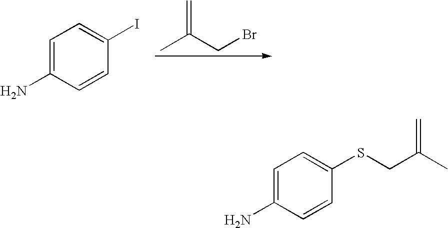

The same procedure as described in Example 23 was repeated but 2-iodoaniline and benzylbromide were replaced with 4-iodobenzenamine 438 mg (2 mmol) and 3-bromo-2-methylprop-1-ene 270 mg (2 mmol), respectively. After purification, the title compound 333 mg (yield: 93%) was obtained.

¹H-NMR (300 MHz, CDCl₃) δ 7.21 (dt, 2H), 6.59 (dt, 2H), 4.67 (m, 2H), 3.65 (bs, 2H), 3.35 (s, 2H), 1.83 (s, 3H).

¹³C-NMR (75.5 MHz, CDCl₃) δ 146.2, 141.6, 134.7, 123.5, 115.6, 113.8, 44.8, 21.1. HRMS (EI) Calcd for C₁₀H₁₃NS (M⁺) 179.0769, found 179.0768.

### Example 31

### Preparation of 2-(4-amineophenylthio)-1-phenylethanone

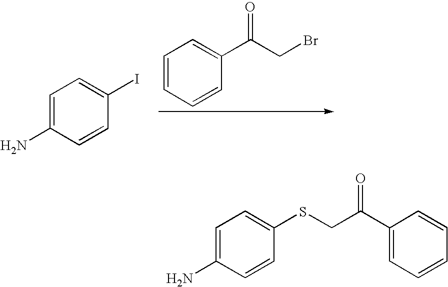

The same procedure as described in Example 23 was repeated but 2-iodoaniline and benzylbromide were replaced with 4-iodobenzenamine 438 mg (2 mmol) and 2-bromo-1-phenylethanone 498 mg (2 mmol), respectively. After purification, the title compound 448 mg (yield: 92%) was obtained.

¹H-NMR (300 MHz, DMSO-d₆) δ 7.91 (m, 2H), 7.56 (m, 1H), 7.44 (m, 2H), 7.22 (dt, 2H), 6.57 (dt, 2H), 4.07 (s, 2H), 3.76 (bs, 2H)

¹³C-NMR (75.5 MHz, DMSO-d₆) δ 194.7, 147.1, 135.8, 135.5, 133.4, 129.0, 128.8, 121.5, 115.8, 43.5.

### Example 32

### Preparation of t-butyl 2-(aminophenylthio)acetate

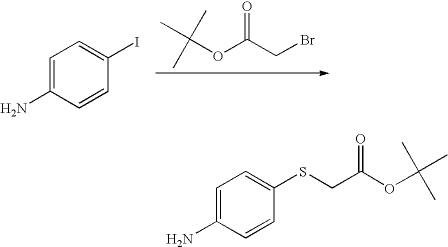

The same procedure as described in Example 23 was repeated but 2-iodoaniline and benzylbromide were replaced with 4-iodobenzenamine 438 mg (2 mmol) and t-butyl 2-bromoacetate 390 mg (2 mmol), respectively. After purification, the title compound 454 mg (yield: 95%) was obtained.

¹H-NMR (300 MHz, DMSO-d₆) δ 7.29 (dt, 2H), 6.60 (dt, 2H), 3.74 (bs, 2H), 3.37 (s, 2H), 1.40 (s, 9H).

¹³C-NMR (75.5 MHz, DMSO-d₆) δ 169.7, 147.0, 135.0, 122.5, 115.8, 81.8, 40.6, 28.3.

### Example 33

### Preparation of 4-(5-phenylpentylthio)benzenamine

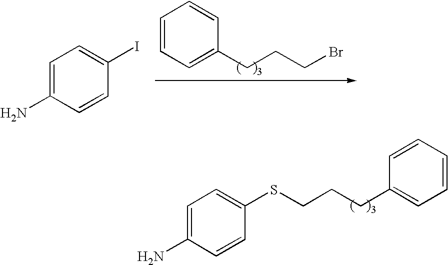

The same procedure as described in Example 23 was repeated but 2-iodoaniline and benzylbromide were replaced with 4-iodobenzenamine 438 mg (2 mmol) and 1-(5-bromopentyl)benzen 454 mg (2 mmol), respectively. After purification, the title compound 456 mg (yield: 84%) was obtained.

¹H-NMR (300 MHz, CDCl₃) δ 7.29˜7.11 (m, 7H), 6.60 (dt, 2H), 3.50 (bs, 2H), 2.75 (t, 2H), 2.58 (t, 2H), 1.62˜1.53 (m, 4H), 1.47˜1.39 (m, 2H).

¹³C-NMR (75.5 MHz, CDCl₃) δ 145.9, 142.8, 134.0, 129.5, 128.6, 128.4, 125.8, 115.8, 36.5, 36.0, 31.2, 29.5, 28.5.

### Example 34

### Preparation of 4-(cyclohexylmethylthio)benzenamine

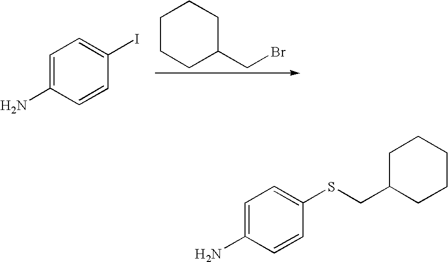

The same procedure as described in Example 23 was repeated but 2-iodoaniline and benzylbromide were replaced with 4-iodobenzenamine 438 mg (2 mmol) and (bromomethyl)cyclohexane 354 mg (2 mmol), respectively. After purification, the title compound 359 mg (yield: 81%) was obtained.

¹H-NMR (300 MHz, CDCl₃) δ 7.42 (d, 2H, J=8.8 Hz), 7.21 (dt, 2H), 6.60 (dt, 2H), 3.66 (bs, 2H), 2.67 (d, 2H), 1.86 (d, 2H), 1.76˜1.57 (m, 3H), 1.54˜1.36 (m, 1H) 1.30˜1.08 (m, 3H), 1.02˜0.85 (m, 2H).

¹³C-NMR (75.5 MHz, CDCl₃) δ 145.7, 139.0, 133.5, 115.8, 115.1, 44.1, 37.7, 32.9, 26.6, 26.3.

### Example 35

### Preparation of 1-(4-aminopentylthio)hex-5-en-2-ol

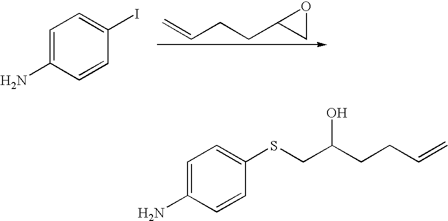

The same procedure as described in Example 23 was repeated but 2-iodoaniline and benzylbromide were replaced with 4-iodobenzenamine 438 mg (2 mmol) and 2-(but-3-enyl)oxiran 196 mg (2 mmol), respectively. After purification, the title compound 389 mg (yield: 87%) was obtained.

¹H-NMR (300 MHz, CDCl₃) δ 7.26 (dt, 2H), 6.61 (dt, 2H), 5.79 (m, 1H), 4.98 (m, 2H), 3.73 (bs, 2H), 3.59 (m, 1H), 2.97 (dd, 1H), 2.68 (dd, 1H), 2.59 (bs, 1H), 2.24 ˜2.05 (m, 2H), 1.66˜1.50 (m, 3H)

¹³C-NMR (75.5 MHz, CDCl₃) δ 146.6, 138.4, 134.6, 122.1, 115.9, 115.0, 68.7, 44.9, 35.2, 30.2.

### Example 36

### Preparation of 4-benzylsulfanyl-benzylamine

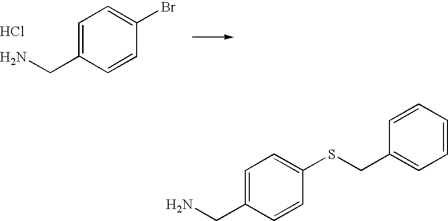

4-bromobenzylamine 445 mg (2 mmol) was completely dissolved in dry tetrahydrofuran 20 ml under nitrogen atmosphere. To the mixture, isopropylmagnesium chloride 3.0 ml (2.0 M-ether, 3.0 equivalent) was slowly added at 0° C. After 10 minutes at room temperature, the mixture was cooled to −78° C. tert-Butyl lithium 1.18 ml (1.7 M-pentane, 2.0 equivalent) was slowly added for 1 minute. After stirring additional 10 minutes, sulfur powder 64 mg (2 mmol, 1.0 equivalent) was added at once at the same temperature. Reaction was carried out so that the temperature of overall reaction mixture was raised to room temperature in 25 minutes. Again, the mixture was cooled to 0° C. Benzylbromide 236 μl (2 mmol, 1.0 equivalent) was slowly added to the mixture. After 20 minutes at room temperature, when the reaction was completed, aqueous ammonium chloride solution was added thereto to quench the reaction. The organic phase was extracted by using ethyl acetate and aqueous sodium chloride solution. After filtration, the solvent was evaporated under reduced pressure. The residue was purified by silica gel column chromatography to obtain the title compound 440 mg (yield: 96%).

¹H-NMR (300 MHz, CDCl₃) δ 7.28˜7.18 (m, 9H), 4.09 (s, 2H), 3.82 (s, 2H).

### Example 37

### Preparation of 2-[4-(benzylthio)phenyl]ethanamine

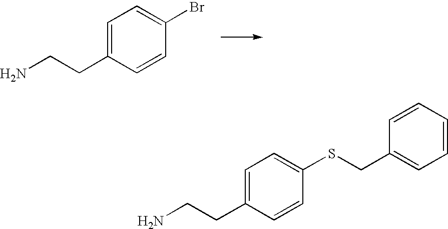

The same procedure as described in Example 36 was repeated but 4-bromobenzylamine was replaced with 2-(4-bromophenyl)ethanamine 400 mg (2 mmol). After purification, the title compound 477 mg (yield: 98%) was obtained.

¹H-NMR (300 MHz, CDCl₃) δ 7.28˜7.21 (m, 7H), 7.09 (d, 2H), 4.09 (s, 2H), 2.94 (t, 2H), 2.70 (t, 2H), 1.75 (bs, 2H)

¹³C-NMR (75.5 MHz, CDCl₃) δ 138.4, 137.8, 134.0, 130.7, 129.6, 129.0, 128.7, 127.3, 43.5, 39.7, 29.9.

### Example 38

### Preparation of t-butyl [4-(2-aminoethyl)phenylthio]-acetate

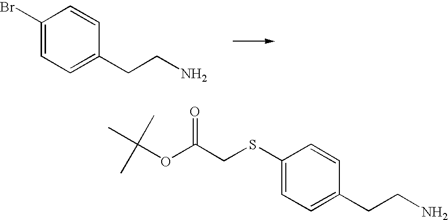

4-bromophenethylamine 400 mg (2 mmol) was completely dissolved in dry tetrahydrofuran 20 ml under nitrogen atmosphere, and the mixture was cooled to 0° C. To the mixture, isopropylmagnesium chloride 2.0 ml (4.0 mmol, 2.0 M-ether, 2.0 equivalent) was slowly added at the same temperature. After 15 minutes, the mixture was cooled to −78° C. tert-Butyl lithium 2.35 ml (4.0 mmol, 1.7 M-pentane, 2.0 equivalent) was slowly added for 1 minute. After 30 minute at the same temperature, sulfur powder 64 mg (2 mmol) dissolved in dry tetrahydrofuran 3.0 ml was added. Reaction was carried out so that the temperature of overall reaction mixture was raised to room temperature in 60 minutes. Again, the mixture was cooled to 0° C. tert-Butylbromoacetate 296 μl (2 mmol, 1.0 equivalent) was slowly added to the mixture. After 20 minutes at room temperature, the solvent was removed under reduced pressure. The organic phase was extracted by aqueous ammonium chloride solution (20 μl) and ethylacetate (3×20 μl). Moisture contained in the organic phase was removed by using magnesium sulfate. After filtration, the solvent was evaporated under reduced pressure. The residue was purified by silica gel column chromatography that using dichloromethane included 3% ammonia water and 10% methanol to obtain the title compound 504 mg (yield 94%).

¹H-NMR (300 MHz, CDCl₃) δ: 7.36 (d, 2H, J=8.1 Hz), 7.13 (d, 2H, J=8.1 Hz), 3.53 (s, 2H), 2.95 (t, 2H, J=6.9 Hz), 2.71 (t, 2H, J=6.9 Hz), 1.40 (s, 9H), 1.31 (br s, 2H).

¹³C-NMR (75.5 MHz, CDCl₃) δ: 169.3, 139.2, 130.9, 129.8, 120.4, 82.2, 43.9, 40.1, 38.5, 28.3.

### Example 39

### Preparation of t-butyl 2[4-(2-aminoethyl)phenylthio]-2-methylpropionate

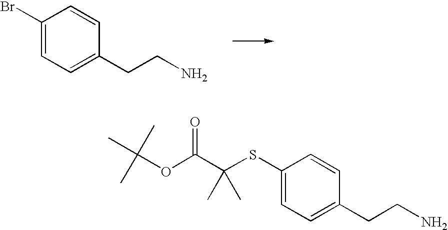

4-bromophenethylamine 397 mg (2 mmol) was completely dissolved in dry tetrahydrofuran 20 ml under nitrogen atmosphere, and the mixture was cooled to 0° C. To the mixture, isopropylmagnesium bromide 2.0 ml (4.0 mmol, 2.0 M-ether, 2.0 equivalent) was slowly added at the same temperature. After 15 minutes, the mixture was cooled to −78° C. tert-Butyl lithium 2.35 μl (4.0 mmol, 1.7 M-pentane, 2.0 equivalent) was slowly added for 1 minute. After 30 minute at the same temperature, sulfur powder 64 mg (2 mmol) dissolved in dry tetrahydrofuran 3.0 ml was added. Reaction was carried out so that the temperature of overall reaction mixture was raised to room temperature. After 60 minutes, the solvent was removed under reduced pressure. After calcium hydroxide 108 mg (2.0 mmol) was added, t-butyl-2-bromoisobutylate 373 μl (2.0 mmol) was added. The reaction mixture was heated with reflux for 1 hour at 80° C., and then cooled to room temperature. After the solvent was removed under reduced pressure, the organic phase was extracted by aqueous ammonium chloride solution (20 μl) and ethylacetate (3×20 μl). Moisture contained in the organic phase was removed by using magnesium sulfate. After filtration, the solvent was evaporated under reduced pressure. The residue was purified by silica gel column chromatography that using dichloromethane included 3% ammonia water and 10% methanol to obtain the title compound 545 mg (yield 92%).

¹H-NMR (300 MHz, CDCl₃) δ: 7.45 (d, 2H, J=88.0 Hz), 7.16 (d, 2H, J=8.0 Hz), 2.97 (t, 2H, J=7.0 Hz), 2.76 (t, 2H, J=7.0 Hz), 1.44 (s, 6H), 1.43 (s, 9H), 1.32 (br s, 2H).

¹³C-NMR (75.5 MHz, CDCl₃) δ: 173.5, 141.5, 137.4, 129.8, 129.4, 129.2, 128.9, 81.3, 51.7, 43.8, 40.2, 28.3, 26.5.

### HREIMS: C₁₆H₂₅NO₂S

Calculated value 295.1606,

Measured value 295.1605.

### Example 40

### Preparation of 2-benzylsulfanylphenol

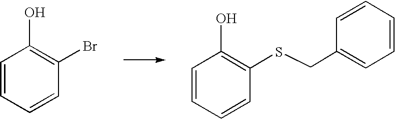

2-bromophenol 232 μl (2 mmol) was completely dissolved in dry tetrahydrofuran 15 ml under nitrogen atmosphere, and the mixture was cooled to 0° C. To the mixture, isopropylmagnesium chloride 1.0 ml (2.0 mmol, 2.0 M-ether, 2.0 equivalent) was slowly added at the same temperature. After 10 minutes, the mixture was cooled to −78° C. tert-Butyl lithium 2.35 μl (4.0 mmol, 1.7 M-pentane, 2.0 equivalent) was slowly added for 1 minute. After 30 minutes at the same temperature, sulfur powder 64 mg (2 mmol) dissolved in dry tetrahydrofuran 3.0 ml was added. Reaction was carried out so that the temperature of overall reaction mixture was raised to room temperature in 30 minutes. Again, the mixture was cooled to 0° C. Benzylbromide 236 μl (2 mmol, 1.0 equivalent) was slowly added to the mixture. After 20 minutes at room temperature, aqueous ammonium chloride solution was added thereto to quench the reaction. The organic phase was extracted by ethylacetate and aqueous sodium chloride solution. Moisture contained in the organic phase was removed by using magnesium sulfate. After filtration, the solvent was evaporated under reduced pressure. The residue was purified by silica gel column chromatography to obtain the title compound 504 mg (yield: 94%).

¹H-NMR (300 MHz, CDCl₃) δ: 7.23 (m, 5H), 7.08 (m, 2H), 6.92 (d, 1H, J=7.8 Hz), 6.79 (t, 1H, J=15.1 and 7.6 Hz), 6.54 (br s, 1H), 3.84 (br s, 2H).

¹³C-NMR (75.5 MHz, CDCl₃) δ: 157.3, 137.8, 136.6, 132.2, 131.6, 129.4, 128.9, 128.7, 127.6, 122.0, 120.8, 118.4, 116.4, 114.9, 41.6.

### Example 41

### Preparation of 3-benzylsulfanylphenol

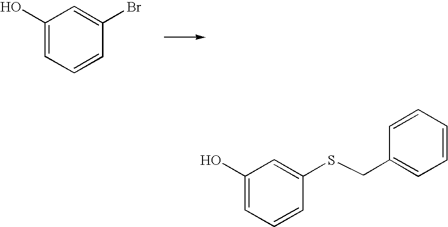

The same procedure as described in Example 40 was repeated but 2-bromophenol was replaced with 3-bromophenol 346 mg (2 mmol). After purification, the title compound 338 mg (yield: 78%) was obtained.

¹H-NMR (300 MHz, CDCl₃) δ: 7.26 (m, 5H), 7.10 (t, 1H, J=16.0 and 8.0 Hz) 6.85 (d, 1H, J=7.8 Hz), 6.77 (t, 1H, J=3.9 and 1.9 Hz), 6.63 (dd, 1H, J=8.1 and 2.4 Hz), 5.46 (br s, 1H), 4.09 (s, 2H).

¹³C-NMR (75.5 MHz, CDCl₃) δ: 155.9, 138.2, 137.4, 130.1, 129.0, 128.7, 127.4, 121.8, 116.2, 113.6, 38.7.

### Example 42

### Preparation of 4-benzylsulfanylphenol

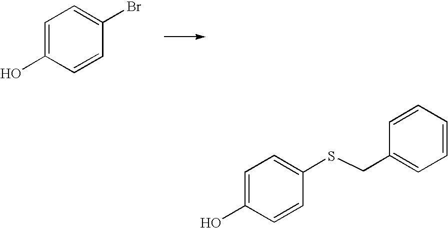

The same procedure as described in Example 40 was repeated but 2-bromophenol was replaced with 4-bromophenol 346 μl (2 mmol). After purification, the title compound 372 mg (yield: 86%) was obtained.

¹H-NMR (300 MHz, CDCl₃) δ: 7.26˜7.16 (m, 7H), 6.69 (d, 2H, J=8.4 Hz), 5.29 (br s, 1H), 3.97 (s, 2H).

¹³C-NMR (75.5 MHz, CDCl₃) δ: 155.4, 138.2, 134.5, 129.1, 128.6, 127.2, 126.3, 116.1, 41.4.

### Example 43

### Preparation of 4-benzylsulfanyl-2,6-dimethylphenol

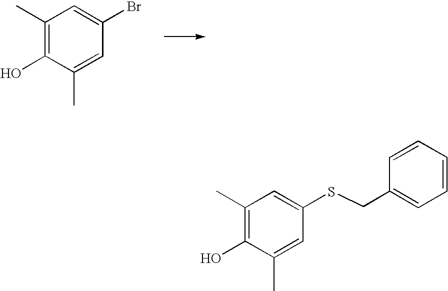

The same procedure as described in Example 40 was repeated but 2-bromophenol was replaced with 4-bromo-2,6-dimethylphenol 402 mg (2 mmol). After purification, the title compound 303 mg (yield: 62%) was obtained.

¹H-NMR (300 MHz, CDCl₃) δ: 7.29˜7.21 (m, 7H), 6.90 (s, 1H), 3.98 (s, 2H), 2.18 (s, 6H).

### Example 44

### Preparation of 4-benzylsulfanyl-2-chlorophenol

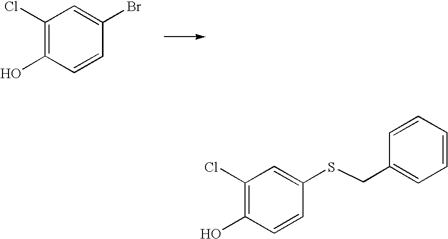

The same procedure as described in Example 40 was repeated but 2-bromophenol was replaced with 4-bromo-2-chlorophenol 415 mg (2 mmol). After purification, the title compound 411 mg (yield: 82%) was obtained.

¹H-NMR (300 MHz, CDCl₃) δ: 7.28˜7.19 (m, 6H), 7.12 (dd, 1H, J=8.5 and 2.2 Hz), 6.89 (d, 1H, J=8.5 Hz) 5.53 (br s, 1H), 3.99 (s, 2H).

### Example 45

### Preparation of 4-benzylsulfanyl-4-fluorophenol

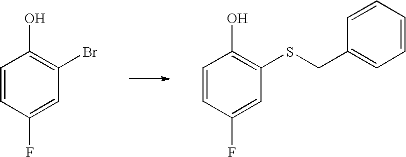

The same procedure as described in Example 40 was repeated but 2-bromophenol was replaced with 4-fluoro-2-bromophenol 382 mg (2 mmol). After purification, the title compound 244 mg (yield: 52%) was obtained.

¹H-NMR (300 MHz, CDCl₃) δ: 7.25 (m, 3H), 7.08 (m, 2H), 6.97˜6.86 (m, 3H), 6.27 (s, 1H), 3.85 (s, 2H).

### Example 46

### Preparation of 2-(pent-2-ynylsulfanyl)-4-fluorophenol

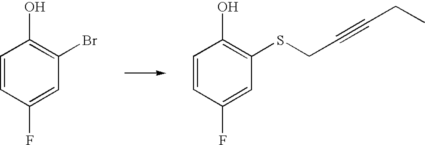

The same procedure as described in Example 40 was repeated but 2-bromophenol and benzylbromide were replaced with 2-bromo-4-fluorophenol 382 mg (2 mmol) and 1-bromo-2-pentyne 204 μl (2 mmol, 1.0 equivalent), respectively. After purification, the title compound 374 mg (yield: 89%) was obtained.

¹H-NMR (300 MHz, CDCl₃) δ: 7.24-7.28 (m, 1H), 6.91-7.04 (m, 2H), 6.56 (s, 1H), 3.40 (t, 2H, J=2.34 Hz), 2.16 (m, 2H), 1.07 (t, 3H, J=7.5 Hz).

¹³C-NMR (75.5 MHz, CDCl₃) δ: 157.99, 154.80, 154.18, 154.15, 122.62, 122.32. 119.02, 118.91, 118.72, 115.97, 115.87, 87.40, 74.67, 25.82, 14.02, 12.82

### Example 47

### Preparation of 2-(5-phenylpentylsulfanyl)-4-fluorophenol

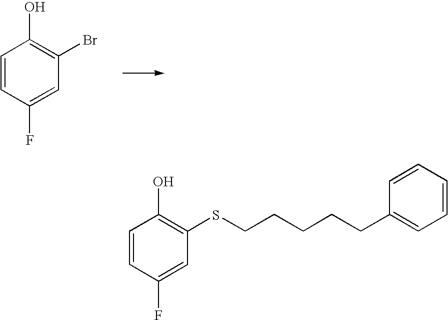

The same procedure as described in Example 40 was repeated but 2-bromophenol and benzylbromide were replaced with 2-bromo-4-fluorophenol 382 mg (2 mmol) and 5-phenylpentyl bromide 375 μl (2 mmol, 1.0 equivalent), respectively. After purification, the title compound 482 mg (yield: 83%) was obtained.

¹H-NMR (300 MHz, CDCl₃) δ: 7.11-7.25 (m, 6H), 6.90-6.94 (m, 2H), 6.46 (s, 1H), 2.67 (t, 2H, J=7.27 Hz), 2.56 (t. 2H, J=7.45 Hz), 1.51-1.63 (m, 4H), 1.32-1.43 (m, 2H).

¹³C-NMR (75.5 MHz, CDCl₃) δ: 158.17, 154.98, 153.57, 153.54, 142.71, 128.82, 128.76, 126.20, 121.81, 121.51, 120.49, 120.39, 118.18, 117.88, 115.83, 115.73, 37.01, 36.15, 31.31, 29.92, 28.57

### Example 48

### Preparation of 2-(cyclohexylmethylsulfanyl)-4-fluorophenol

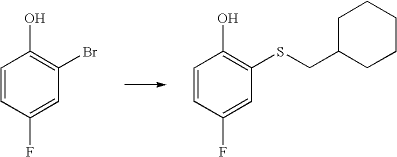

The same procedure as described in Example 40 was repeated but 2-bromophenol and benzylbromide were replaced with 2-bromo-4-fluorophenol 382 mg (2 mmol) and bromomethyl cyclohexane 277 μl (2 mmol, 1.0 equivalent), respectively. After purification, the title compound 389 mg (yield: 81%) was obtained.

¹H-NMR (300 MHz, CDCl₃) δ: 7.16-7.20 (m, 1H), 6.90-7.00 (m, 2H), 6.50 (s, 1H), 2.64 (d, 2H, J=6.89 Hz), 1.85-1.89 (m, 2H), 1.66-1.77 (m, 3H), 1.46 (m, 1H), 1.17-1.28 (m, 3H), 0.95-0.99 (m, 2H).

¹³C-NMR (75.5 MHz, CDCl₃) δ: 158.15, 154.97, 153.26, 153.23, 121.52, 121.41, 121.30, 121.21, 117.85, 115.75, 115.64, 44.77, 38.09, 32.91, 26.65, 26.31.

### Example 49

### Preparation of 4-(2-(1,3-dioxolan-2-yl)ethylsulfanyl)phenol

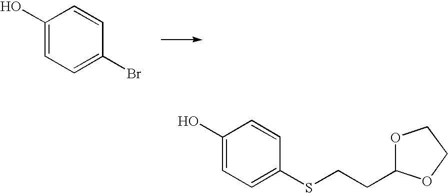

The same procedure as described in Example 40 was repeated but 2-bromophenol and benzylbromide were replaced with 4-bromophenol 346 mg (2 mmol) and 2-(2-bromomethyl)-1,3-dioxolan 235 μl (2 mmol, 1.0 equivalent), respectively. After purification, the title compound 353 mg (yield: 78%) was obtained.

¹H-NMR (300 MHz, CDCl₃) δ: 7.26-7.40 (m, 2H), 6.71-6.77 (m, 2H), 5.60 (br, 1H), 4.97-5.00 (m, 1H), 3.84-4.02 (m, 4H), 2.90 (m, 2H), 1.93 (m, 2H).

¹³C-NMR (75.5 MHz, CDCl₃) δ: 155.60, 134.06, 126.15, 116.47, 103.58, 65.36, 34.04, 30.52.

### Example 50

### Preparation of 2-(2-hydroxyhex-5-enylsulfanyl)-4-fluorophenol

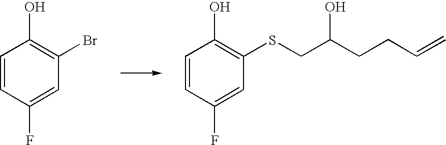

The same procedure as described in Example 40 was repeated but 2-bromophenol and benzylbromide were replaced with 2-bromo-4-fluorophenol 382 mg (2 mmol) and 1,2-epoxy-5-hexen 228 μl (2 mmol, 1.0 equivalent), respectively. After purification, the title compound 411 mg (yield: 85%) was obtained.

¹H-NMR (300 MHz, CDCl₃) δ: 7.39 (br, 1H), 7.18-7.22 (m, 1H), 6.89-7.00 (m, 2H), 5.73-5.82 (m, 1H), 4.96-5.05 (m, 2H), 3.70 (br, 1H), 3.05 (br, 1H), 2.93-2.99 (m, 1H), 2.71-2.78 (m, 1H), 2.10-2.20 (m, 2H), 1.58-1.66 (m, 2H).

¹³C-NMR (75.5 MHz, CDCl₃) δ: 158.06, 154.87, 153.88, 153.85, 138.06, 122.16, 121.86, 120.43, 120.32, 118.34, 118.04, 116.73, 116.62, 115.85, 70.02, 44.19, 35.59, 30.3.

### Example 51

### Preparation of (2-(benzylsulfanyl)phenyl)methanol

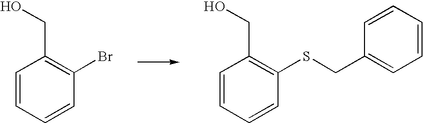

The same procedure as described in Example 40 was repeated but 2-bromophenol was replaced with 2-bromobenzylalcohol 374 mg (2 mmol). After purification, the title compound 432 mg (yield: 94%) was obtained.

¹H-NMR (300 MHz, CDCl₃) δ: 7.15-7.34 (m, 9H), 4.58 (s, 2H), 4.01 (s, 1H), 2.29 (br, 1H).

¹³C-NMR (75.5 MHz, CDCl₃) δ: 142.09, 137.85, 134.51, 132.11, 129.25, 128.98, 128.75, 128.66, 127.97, 127.75, 63.87, 40.24, 31.62.

### Example 52

### Preparation of (3-(benzylsulfanyl)phenyl)methanol

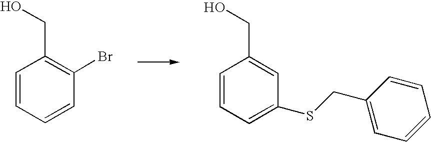

The same procedure as described in Example 40 was repeated but 2-bromophenol was replaced with 3-bromobenzylalcohol 374 mg (2 mmol). After purification, the title compound 409 mg (yield: 89%) was obtained.

¹H-NMR (300 MHz, CDCl₃) δ: 7.10-7.26 (m, 9H), 4.54 (s, 2H), 4.09 (s, 2H), 2.05 (br, 1H).

¹³C-NMR (75.5 MHz, CDCl₃) δ: 142.04, 137.75, 137.14, 129.43, 129.31, 129.11, 128.93, 128.43, 127.64, 125.27, 65.28, 39.25.

### Example 53

### Preparation of (4-(benzylsulfanyl)phenyl)methanol

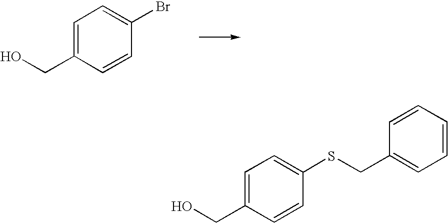

The same procedure as described in Example 40 was repeated but 2-bromophenol was replaced with 4-bromobenzylalcohol 374 mg (2 mmol). After purification, the title compound 426 mg (yield: 92%) was obtained.

¹H-NMR (300 MHz, CDCl₃) δ: 7.29-7.20 (m, 9H), 4.60 (s, 2H), 4.09 (s, 2H), 1.87 (br, 1H).

¹³C-NMR (75.5 MHz, CDCl₃) δ: 139.49, 137.80, 135.99, 130.39, 129.22, 128.97, 128.92, 127.92, 127.62, 65.25, 39.4.

### Example 54

### Preparation of (4-(pent-2-ynylsulfanyl)phenyl)methanol

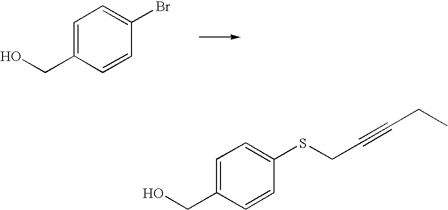

The same procedure as described in Example 40 was repeated but 2-bromophenol and benzylbromide were replaced with 4-bromo-benzylalcohol 374 mg (2 mmol) and 1-bromo-2-pentine 204 μl (2 mmol, 1.0 equivalent), respectively. After purification, the title compound 375 mg (yield: 91%) was obtained.

¹H-NMR (300 MHz, CDCl₃) δ: 7.26-7.42 (m, 4H), 4.63 (s, 2H), 3.59 (m, 2H), 2.16 (m, 1H), 2.02 (br, 1H), 1.08 (t, 3H, J=7.44 Hz).

¹³C-NMR (75.5 MHz, CDCl₃) δ: 139.79, 135.30, 130.49, 127.88, 86.00, 75.25, 65.21, 23.63, 14.22, 12.90.

### Example 55

### Preparation of tert-butyl 2-((4-(hydroxymethyl)phenyl)sulfanyl)acetate

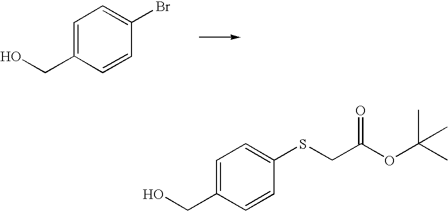

The same procedure as described in Example 40 was repeated but 2-bromophenol and benzylbromide were replaced with 4-bromo-benzylalcohol 374 mg (2 mmol) and t-butyl bromoacetate 296 μl (2 mmol, 1.0 equivalent), respectively. After purification, the title compound 452 mg (yield: 89%) was obtained.

¹H-NMR (300 MHz, CDCl₃) δ: 7.23-7.41 (m, 4H), 4.59 (s, 2H), 3.51 (s, 2H), 2.63 (br, 1H), 1.39 (s, 9H).

¹³C-NMR (75.5 MHz, CDCl₃) δ: 169.31, 140.15, 134.65, 130.39, 127.88, 82.41, 64.90, 38.19, 28.27.

### Example 56

### Preparation of (4-(2-(1,3-dioxolan-2-yl)ethylsulfanyl)phenyl)methanol

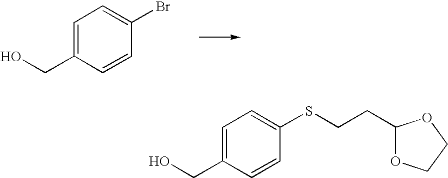

The same procedure as described in Example 40 was repeated but 2-bromophenol and benzylbromide were replaced with 4-bromo-benzylalcohol 374 mg (2 mmol) and 2-(2-bromomethyl)-1,3-dioxolan 235 μl (2 mmol, 1.0 equivalent), respectively. After purification, the title compound 360 mg (yield: 75%) was obtained.

¹H-NMR (300 MHz, CDCl₃) δ: 7.19-7.36 (m, 4H), 4.96 (t, 1H, J=4.46 Hz), 4.60 (s, 2H), 3.81-3.97 (m, 4H) 2.97-3.02 (m, 2H), 2.29 (br, 1H), 1.97 (m, 1H).

¹³C-NMR (75.5 MHz, CDCl₃) δ: 139.22, 135.79, 129.70, 128.02, 103.42, 65.38, 65.14, 33.86, 28.28.

### Example 57

### Preparation of 1-((4-(hydroxymethyl)phenyl)sulfanyl)hex-5-en-2-ol

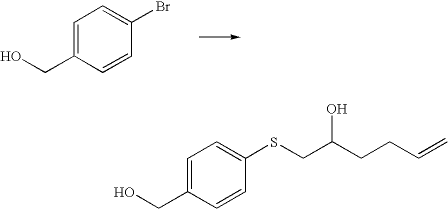

The same procedure as described in Example 40 was repeated but 2-bromophenol and benzylbromide were replaced with 4-bromo-benzylalcohol 374 mg (2 mmol) and 1,2-epoxy-5-hexen 228 μl (2 mmol, 1.0 equivalent), respectively. After purification, the title compound 395 mg (yield: 83%) was obtained.

¹H-NMR (300 MHz, CDCl₃) δ: 7.22-7.34 (m, 4H), 5.78 (m, 1H), 4.94-5.05 (m, 2H), 4.59 (s, 2H), 3.68 (m, 1H), 3.06-3.12 (m, 1H), 2.81-2.88 (m, 3H), 2.15 (m, 2H), 1.60 (m, 2H).

¹³C-NMR (75.5 MHz, CDCl₃) δ: 139.81, 138.40, 134.91, 130.40, 128.08, 115.45, 69.44, 64.88, 42.39, 35.53, 30.28.

### Example 58

### Preparation of 2-(2-(benzylsulfanyl)phenyl)ethanol

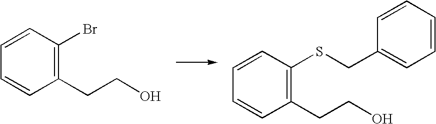

The same procedure as described in Example 40 was repeated but 2-bromophenol was replaced with 2-bromo-phenethylalcohol 402 mg (2 mmol). After purification, the title compound 444 mg (yield: 91%) was obtained.

¹H-NMR (300 MHz, CDCl₃) δ: 7.13-7.36 (m, 9H), 4.08 (s, 2H), 3.77 (dd, 2H, J=6.66 Hz, 6.69 Hz), 2.96 (dd, 2H, J=6.66 Hz, 6.69 Hz), 1.36 (br, 1H).

¹³C-NMR (75.5 MHz, CDCl₃) δ: 138.85, 137.36, 135.84, 130.56, 130.42, 129.00, 128.64, 127.40, 127.37, 126.74, 62.80, 39.44, 37.32.

### Example 59

### Preparation of 2-(3-(benzylsulfanyl)phenyl)ethanol

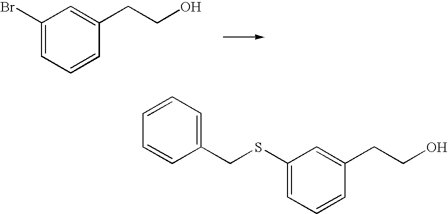

The same procedure as described in Example 40 was repeated but 2-bromophenol was replaced with 3-bromo-phenethylalcohol 402 mg (2 mmol). After purification, the title compound 439 mg (yield: 90%) was obtained.

¹H-NMR (300 MHz, CDCl₃) δ: 7.03-7.28 (m, 9H), 4.10 (s, 2H), 3.79 (m, 2H), 2.78 (t, 2H, J=6.49 Hz), 1.34 (br, 1H).

¹³C-NMR (75.5 MHz, CDCl₃) δ: 139.41, 137.52, 136.55, 130.48, 129.48, 129.10, 128.96, 128.63, 128.56, 127.88, 127.29, 127.24, 63.47, 39.07, 39.00.

### Example 60

### Preparation of 2-(4-(benzylsulfanyl)phenyl)ethanol

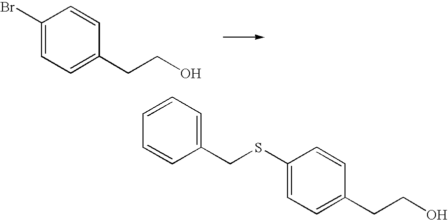

The same procedure as described in Example 40 was repeated but 2-bromophenol was replaced with 4-bromo-phenethylalcohol 402 mg (2 mmol). After purification, the title compound 439 mg (yield: 90%) was obtained.

¹H-NMR (300 MHz, CDCl₃) δ: 7.10-7.28 (m, 9H), 4.09 (s, 2H), 3.83 (m, 2H), 2.82 (t, 2H, J=6.51 Hz), 1.37 (br, 1H).

¹³C-NMR (75.5 MHz, CDCl₃) δ: 137.94, 137.33, 134.58, 130.77, 129.95, 129.22, 128.88, 127.56, 63.91, 39.73, 39.10.

### Example 61

### Preparation of 6-benzylsulfanyl-naphthalen-2-ol

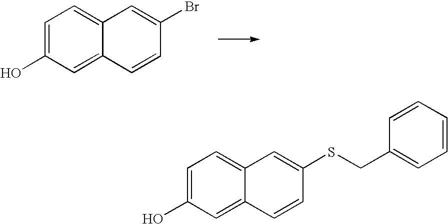

The same procedure as described in Example 40 was repeated but 2-bromophenol was replaced with 6-bromo-2-naphtol 446 mg (2 mmol). After purification, the title compound 490 mg (yield: 92%) was obtained.

¹H-NMR (300 MHz, CDCl₃) δ: 7.67 (d, 1H, J=1.6 Hz), 7.60 (d, 1H, J=9.3 Hz), 7.55 (d, 1H, J=8.7 Hz), 5.13 (br s, 1H), 4.15 (s, 2H).

¹³C-NMR (75.5 MHz, CDCl₃) δ: 153.7, 137.8, 133.5, 130.9, 129.5, 129.4, 129.1, 128.7, 127.4, 127.1, 118.5, 109.7, 39.9.

### Example 62

### Preparation of t-butyl(4-hydroxyphenylsulfanyl)acetate

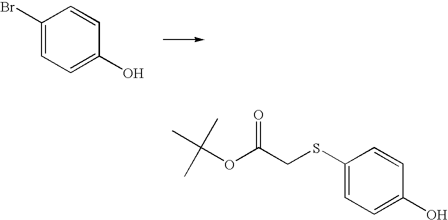

The same procedure as described in Example 40 was repeated but 2-bromophenol and benzylbromide were replaced with 4-bromophenol 346 mg (2 mmol) and tert-butyl bromoacetate 295 μl (2 mmol, 1.0 equivalent), respectively. After purification, the title compound 447 mg (yield: 93%) was obtained.

¹H-NMR (300 MHz, CDCl₃) δ: 7.31 (d, 2H, J=8.8 Hz), 6.67 (d, 2H, J=8.8 Hz), 6.33 (br s, 1H), 3.39 (s, 2H), 1.39 (s, 9H).

### Example 63

### Preparation of 5-[4-(tert-butyldimethylsilanyloxy)-3-methyl-phenylsulfanyl-4-methyl-2-[(4-trifluoromethyl)phenyl]thiazol

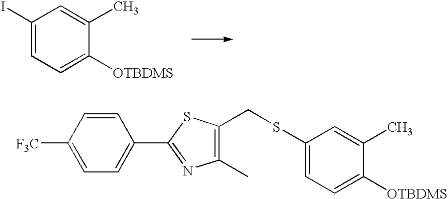

4-iodine-2-methyl-phenoxy-tert-butyldimethyl silane 500 mg (1.74 mmol) was completely dissolved in dry tetrahydrofuran 40 ml under nitrogen atmosphere, and the mixture was cooled to −78° C. To the mixture, n-butyl lithium 1.09 ml (1.6M in hexane, 1.0 equivalent) was slowly added for 1 minute. After stirring additional 10 minutes, sulfur powder 55.7 mg (1.74 mmol, 1.0 equivalent) was added at once at the same temperature. After stirring the mixture at the same temperature for additional 10 minutes to completely dissolve sulfur, 5-chloromethyl-4-methyl-2-[(4-trifluoromethyl)phenyl]thiazol 420 mg (1.74 mmol, 1.0 equivalent) was added at once. Reaction was carried out so that the temperature of overall reaction mixture was raised to room temperature in 60 minutes. Aqueous ammonium chloride solution was added thereto to quench the reaction. The organic phase was extracted by using ethyl acetate and aqueous sodium chloride solution. Moisture contained in the organic phase was removed by using magnesium sulfate. After filtration, the solvent was evaporated under reduced pressure. The residue was purified by silica gel column chromatography to obtain the title compound 730 mg (yield: 84.6%).

¹H-NMR (300 MHz, CDCl₃) δ: 7.97 (d, 2H, J=8.0 Hz), 7.65 (d, 2H, J=8.2 Hz), 7.17 (d, 1H, J=1.8 Hz), 7.07 (dd, 1H, J=8.2 and 2.3 Hz), 6.67 (d, 1H, J=8.3 Hz), 4.10 (s, 2H), 2.20 (s, 3H), 2.15 (s, 3H), 1.00 (s, 9H), 0.20 (s, 6H).

¹³C-NMR (75.5 MHz, CDCl₃) δ: 163.4, 154.9, 151.8, 136.8, 132.6, 130.4, 129.6 (q, J=32 Hz), 126.8, 126.2 (q, J=4 Hz), 125.2, 119.6, 33.0, 26.1, 18.7, 17.1, 15.2,-3.9.

### Example 64

### Preparation of 2-methyl-4-[[[4-methyl-2-[(4-trifluoromethyl)phenyl]thiazol-5-yl]methyl]sulfanyl]phenol

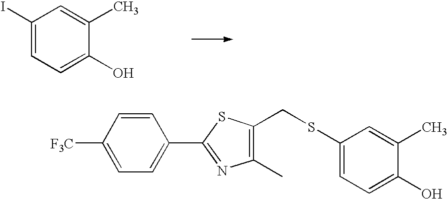

4-iodine-2-methylphenol 11.7 g (50.0 mmol) was completely dissolved in dry tetrahydrofuran 400 ml under nitrogen atmosphere, and the mixture temperature was maintained at 0° C. To the mixture, isopropylmagnesium chloride 27.5 ml (2.0 M-ether, 1.1 equivalent) was slowly added at the same temperature. After 10 minutes, the mixture was cooled to −78° C. tert-Butyl lithium 64.7 μl (1.7 M-pentane, 2.2 equivalent) was slowly added. After 20 minutes, sulfur powder 1.60 g (50 mmol, 1.0 equivalent) dissolved in dry THF 50 ml was slowly added. Reaction was carried out so that the temperature of overall reaction mixture was raised to 0° C. After 60 minutes, 5-chloromethyl-4-methyl-2-[(4-trifluoromethyl)phenyl]thiazol 13.1 g (45.0 mmol, 0.9 equivalent) dissolved in dry THF 40 ml was added at 0° C. After 30 minutes at room temperature, aqueous ammonium chloride solution 500 μl was added thereto to quench the reaction. The organic phase was extracted. Moisture contained in the organic phase was removed by using magnesium sulfate. After filtration, the solvent was evaporated under reduced pressure. The residue was purified by silica gel column chromatography using hexan/ethylacetate (v/v=3/1) to obtain the title compound 16.2 mg (yield: 91%).

¹H-NMR (300 MHz, CDCl₃) δ: 7.96 (d, 2H, J=8.2 Hz), 7.64 (d, 2H, J=8.3 Hz), 7.20 (d, 1H, J=1.8 Hz), 6.97 (dd, 1H, J=8.2 and 2.2 Hz), 6.59 (d, 1H, J=8.2 Hz), 5.52 (br s, 1H), 4.06 (s, 2H), 2.19 (s, 3H), 2.09 (s, 3H).

¹³C-NMR (75.5 MHz, CDCl₃) δ: 164.1, 155.5, 151.7, 137.4, 136.8, 133.6, 131.9 (q, J=33 Hz), 131.8, 131.6, 126.9, 126.4 (q, J=4 Hz), 125.9, 123.8, 115.7, 33.2, 16.2, 14.8.

### Use Possibility at Industry

As described above, according to the process of the invention, alkyl aryl sulfide derivatives represented by Chemical Formula (III) can be prepared in a simple process with high yield.

1. A process of preparing an alkyl aryl sulfide represented by Chemical Formula (III) which comprises substituting a halogen atom of an aryl halogen compound represented by Chemical Formula (I) with an alkyl lithium represented by Chemical Formula (IV), and subsequently reacting with sulfur and a compound represented by Chemical Formula (II): 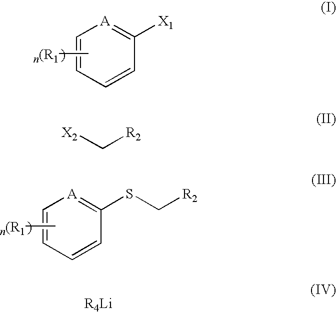
wherein, A represents CH or a nitrogen atom,
X₁ represents a halogen atom,
X₂ represents a halogen atom or a leaving group,
R₁ represents a hydrogen atom, a halogen atom, a C₁-C₇ alkyl group, a C₁-C₇ alkyloxy group, a C₁-C₇ alkylthiooxy group or an aryl group, wherein the alkyl group may be substituted by one or more substituent(s) selected from the group consisting of halogen atoms and a hydroxyl group,
R₂ represents a C₁-C₁₀ alkyl group, an aryl group, a C₁-C₁₀ alkylester group, a C₁-C₁₀ alkylketone group or an arylketone group,
R₄ represents a C₁-C₄ alkyl group, and
n represents an integer of 1 to 3.

2. A process of preparing an alkyl aryl sulfide represented by Chemical Formula (III), which comprises reacting an aryl halogen compound represented by Chemical Formula (I) with an alkyl magnesium halide represented by Chemical Formula (V) to protect the hydrogen-donor substituent, substituting the halogen of the compound (I) with an alkyl lithium represented by Chemical Formula (IV), and subsequently reacting with sulfur and a compound represented by Chemical Formula (II): 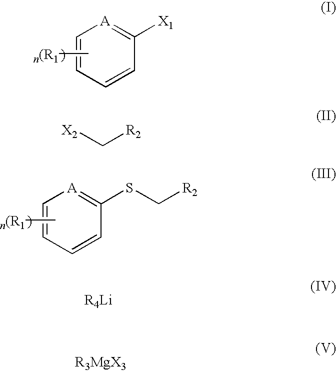
wherein, A represents CH or a nitrogen atom,
X₁ represents a halogen atom,
X₂ represents a halogen atom or a leaving group,
X₃ represents a halogen atom,
R₁ represents hydroxyl group, hydroxymethyl, hydroxyethyl, amine group, aminomethyl, aminoethyl, alkylamine, dialkylamine, carboxylic group, halogen atom or C₁-C₄ alkyl group,
R₂ represents a C₁-C₁₀ alkyl group, an aryl group, a C₁-C₁₀ alkylester group, a C₁-C₁₀ alkylketone group or an arylketone group,
R₃ and R₄ independently represents a C₁-C₄ alkyl group, and
n represents an integer of 1 to 3.

3. A process for preparing an alkyl aryl sulfide represented by Chemical Formula (III) according to claim 2, wherein the alkylmagnesium halide is a compound selected from the group consisting of CH₃MgCl, CH₃MgBr, CH₃MgI, CH₃CH₂MgCl, CH₃CH₂MgBr, CH₃CH₂MgI, CH₃CH₂CH₂MgCl, CH₃CH₂CH₂MgBr, CH₃CH₂CH₂MgI, (CH₃)₂CHMgCl, (CH₃)₂CHMgBr, (CH₃)₂CHMgI, CH₃CH₂CH₂CH₂MgCl, CH₃CH₂CH₂CH₂MgBr, CH₃CH₂CH₂CH₂MgI, C₂H₅CHCH₃MgCl, C₂H₅CHCH₃MgBr, C₂H₅CHCH₃MgI, (CH₃)₃CMgCl, (CH₃)₃CMgBr and (CH₃)₃CMgI.

4. A process of preparing an alkyl aryl sulfide represented by Chemical Formula (III) according to claim 1, wherein the alkyl lithium employed is n-butyl lithium, sec-butyl lithium or tert-butyl lithium, and the amount employed is 1 to 3 equivalants with respect to the compound of Chemical Formula (I).

5. An alkyl aryl sulfide selected from following compounds:
benzyl 2-trifluoromethylphenyl sulfide;
benzyl 2-methoxyphenyl sulfide;
2-bromo-6-(2-[1,3]dioxolan-2-yl-ethylsulfanyl)pyridine;
5-[4-(tert-butyldimethylsilanyloxy)-3-methylphenylsulfanyl]-4-methyl-2-[(4-trifluoromethyl)phenyl]thiazole;
4-benzylsulfanyl-2-methyl-phenylamine;
tert-butyl [4-(2-aminoethyl)phenylthio]acetate;
4-benzylsulfanyl-2,6-dimethylphenol;
4-benzylsulfanyl-2-chlorophenol;
4-benzylsulfanyl-4-fluorophenol;
(4-benzylsulfanylphenyl)methanol;
tert-butyl(4-hydroxyphenylsulfanyl)acetate;
2-methyl-4-[[[4-methyl-2-[(4-trifluoromethyl)phenyl]thiazol-5-yl]methyl]sulfanyl]phenol;
2-(pent-2-ynylsulfanyl)-4-fluorophenol;
2-(5-phenylpentylsulfanyl)-4-fluorophenol;
2-(cyclohexylmethylsulfanyl)-4-fluorophenol;
4-((2-(1,3-dioxolan-2-yl)ethylsulfanyl)phenol;
2-(2-hydroxyhex-5-enylsulfanyl)-4-fluorophenol;
4-((tert-butoxycarbonyl)methylsulfanyl)benzoic acid;
3-(2-(1,3-dioxolan-2-yl)ethylsulfanyl)benzoic acid;
3-(2-hydroxyhex-5-enylsulfanyl)benzoic acid;
2-(4-(benzylsulfanyl)phenyl)ethanol;
2-(3-(benzylsulfanyl)phenyl)ethanol;
1-((4-(hydroxymethyl)phenyl)sulfanyl)hex-5-en-2-ol;
(4-(2-(1,3-dioxolan-2-yl)ethylsulfanyl)phenyl)methanol;
tert-butyl 2-((4-(hydroxymethyl)phenyl)sulfanyl)acetate;
(4-(pent-2-ynylsulfanyl)phenyl)methanol;
4-(benzylthio)-2-bromobenzenamine;
4-(5-phenylpentylthio)benzeneamine;
1-(4-aminopentylthio)hex-5-en-2-ol;
2-[4-(benzylthio)phenyl]ethanamine; and
tert-butyl 2-[4-(2-aminoethyl)phenylthio]-2-methylpropionate.

6. A process of preparing an alkyl aryl sulfide represented by Chemical Formula (III) according to claim 2, wherein the alkyl lithium employed is n-butyl lithium, sec-butyl lithium or tert-butyl lithium, and the amount employed is 1 to 3 equivalants with respect to the compound of Chemical Formula (I).
